SEQUENCE LISTING (17 sequences)

SEQ ID NO: 1 - protein, 30 aa, Homo sapiens
NPMYNAVSNADLMDFKNLLDHLEEKMPLED

SEQ ID NO: 2 - protein, 37 aa, Homo sapiens
EVVPPQVLSEPNEEAGAALSPLPEVPPWTGEVSPAQR

SEQ ID NO: 3 - protein, 20 aa, Homo sapiens
SSDRSALLKSKLRALLTAPR

SEQ ID NO: 4 - protein, 28 aa, Homo sapiens
  DISULFID (7)..(23)
SLRRSSCFGGRMDRIGAQSGLGCNSFRY

SEQ ID NO: 5 - protein, 32 aa, Homo sapiens
  DISULFID (11)..(27)
TAPRSLRRSSCFGGRMDRIGAQSGLGCNSFRY

SEQ ID NO: 6 - protein, 32 aa, Homo sapiens
  DISULFID (10)..(26)
SPKMVQGSGCFGRKMDRISSSSGLGCKVLRRH

SEQ ID NO: 7 - protein, 22 aa, Homo sapiens
  DISULFID (6)..(22)
GLSKGCFGLKLDRIGSMSGLGC

SEQ ID NO: 8 - protein, 15 aa, Dendroaspis angusticeps
PSLRDPRPNAPSTSA

SEQ ID NO: 9 - protein, 37 aa, Artificial Sequence
  Chimeric peptide
GLSKGCFGLKLDRIGSMSGLGCPSLRDPRPNAPSTSA

SEQ ID NO: 10 - protein, 32 aa, Artificial Sequence
  Chimeric peptide
TAPRSLRRSSCFGLKLDRIGSMSGLGCNSFRY

SEQ ID NO: 11 - protein, 17 aa, Homo sapiens
  DISULFID (1)..(17)
CFGLKLDRIGSMSGLGC

SEQ ID NO: 12 - protein, 10 aa, Homo sapiens
TAPRSLRRSS

SEQ ID NO: 13 - protein, 5 aa, Homo sapiens
NSFRY

SEQ ID NO: 14 - protein, 37 aa, Artificial Sequence
  Chimeric peptide
GLSKGCFGRKMDRIGSMSGLGCPSLRDPRPNAPSTSA

SEQ ID NO: 15 - protein, 37 aa, Artificial Sequence
  Chimeric peptide
GLSKGCFGLKLDRISSSSGLGCPSLRDPRPNAPSTSA

SEQ ID NO: 16 - protein, 37 aa, Artificial Sequence
  Chimeric peptide
GLSKGCFGXXXDRIGSMSGLGCPSLRDPRPNAPSTSA

SEQ ID NO: 17 - protein, 37 aa, Artificial Sequence
  Chimeric peptide
GLSKGCFGLKLDRIXXXSGLGCPSLRDPRPNAPSTSA

(19) United States
(12) Patent Application Publication
(10) Pub. No.: US 2015/0080844 A1
(43) Pub. Date: Mar. 19, 2015

(54) THERAPY FOR KIDNEY DISEASE AND/OR HEART FAILURE BY INTRADERMAL INFUSION

(71) Applicant: Medtronic, Inc., Minneapolis, MN (US)

(72) Inventors: Maura Donovan, St. Paul, MN (US); William J. L. van Antwerp, Valencia, CA (US); Chad A. Wieneke, Plymouth, MN (US); Mike Kaytor, Maplewood, MN (US); Eric Grovender, Minneapolis, MN (US)

(73) Assignee: Medtronic, Inc., Minneapolis, MN (US)

(21) Appl. No.: 14/390,227

(22) PCT Filed: Mar. 15, 2013
(86) PCT No.: PCT/US2013/032448

§ 371 Date: Oct. 2, 2014

Related U.S. Application Data
(60) Provisional application No. 61/619,413, filed on Apr. 2, 2012.

(51) Int. Cl. A61M 37/00; A61K 38/22

(52) U.S. Cl. CPC A61M 37/0015; A61K 38/2242; A61M 2037/0023; A61M 2037/0061
USPC 604/505; 514/12.4; 604/67

(57) ABSTRACT

Intradermal delivery devices, systems and methods thereof for the administration of a natriuretic or chimeric peptide are described. The described delivery devices, systems and methods provide for the treatment of pathological conditions such as kidney disease alone, heart failure alone, concomitant kidney disease and heart failure, or cardiorenal syndrome by delivery of a natriuretic or chimeric peptide through a microneedle array using a delivery pump. The described delivery devices, systems and methods can provide for greater availability of a natriuretic or chimeric peptide and improved pharmacokinetics.

### REFERENCE TO SEQUENCE LISTING

This application contains a “Sequence Listing” submitted as an electronic .txt file. The information contained in the Sequence Listing is hereby incorporated by reference.

### FIELD OF THE INVENTION

The invention relates to therapies involving the administration of a natriuretic peptide for the treatment of pathological conditions such as kidney disease alone, heart failure alone, concomitant kidney disease and heart failure, or cardiorenal syndrome. The invention further relates to the field of chronic and acute delivery of a therapeutic composition using a pump in fluid communication with a microneedle array and methods for administering the therapeutic composition including but not limited to intradermal delivery by either a bolus injection or continuous infusion. The non-limiting methods of delivery contemplated by the invention include arrays of microneedles for delivery of a liquid composition to the dermis, pumps for controlling the rate of delivery and local controlled release technology.

### BACKGROUND

Many protein and peptide drugs are delivered by injection. In some instances, the injection is intravenous to a vein or intramuscular or subcutaneous into the lower layers of the skin. However, such techniques typically require the assistance of trained medical professional or hypodermic needles, and are oftentimes unsuitable for home administration. Further, delivery by conventional needle injection by the patient makes self-delivery of a drug by a patient often difficult. One form of treatment for Heart Failure (HF) and Kidney Disease (KD) is delivery of natriuretic peptides that can increase natriuresis and diuresis. However, continuous infusion of the peptide is often required to maintain a stable level of the drug in the blood rather than intermittent bolus injection. Such a treatment requires the insertion of a needle through the skin for an extended period of time which can increase patient discomfort and co plicate home delivery of the drug as required during chronic delivery of a natriuretic peptide in the treatment of HF and/or KD patients.

Peptide-based drugs used to treat HF and KD are also subject to degradation by proteases. Unlike large, multi-chain proteins such as insulin, which have a significant amount of secondary structure that can protect against degradation, small peptide drugs such as natriuretic peptides are susceptible to degradation prior to absorption into the blood stream. The specific sequence of the amino acids forming a particular peptide can also significantly affect the rate of uptake of that peptide into the vascular system as well as its susceptibility to different proteases. For example, peptides can vary in hydrophobicity, which can affect their ability to move from the interstitial fluid through the capillary wall to reach the circulation. As such, peptides can vary in both rate of absorption and bioavailability depending on route of administration. Moreover, the quality and quantity of proteases present in different paths of administration can affect peptides differently depending of the chemical properties of the peptide in question as well as the ability of the peptide to be absorbed into the circulation from surrounding tissues.

Even where a peptide is successfully delivered to a region of the body where it can access the vascular system, proteolytic enzymes in the vasculature and surrounding tissues can hydrolyze the peptides used to treat HF and KD. In particular, atrial natriuretic peptide (ANP) has been demonstrated to exhibit poor bioavailability when administered through subcutaneous bolus administration. Crozier I G, Nicholls M G, Ikram H, Espiner E A, Yandle T, Plasma immunoreactive atrial natriuretic peptide levels after subcutaneous alpha-hANP injection in normal humans. J Cardiovasc Pharmacol 1987; 10:72-75; Osterode W, Nowotny P, Vierhapper H, Waldhausl W. Kinetics of plasma cyclic GMP and atrial natriuretic peptide after intravenous, intramuscular and subcutaneous injection of 50 micrograms hANP in man, Horm Metab Res 1995; 27:100-103.

Hence, there is an unmet need for devices, systems and methods administering natriuretic peptides having improved delivery properties that safely and effectively improve cardiac performance and modulate fluid. There is also a need for delivering natriuretic peptide in a continuous manner with improved bioavailability and absorption characteristics. There is an unmet need for monitored, home-administration systems and devices for chronic and acute delivery of a natriuretic peptide using a combination pump and microneedle array for intradermal delivery. There is also an unmet need for devices, systems and methods that improve the quality of life and outcomes of patients having acute and worsening decompensated HF and KD wherein the devices, systems and methods are easy to use, convenient, experience less pain, self-administrable, and suitable for home use.

### SUMMARY OF THE INVENTION

The disclosure provided herein is directed to a study of continuous intradermal (ID) administration of natriuretic peptide hormones such as Atrial Natriuretic Peptide (ANP) vessel dilator (VD) kaliuretic peptide (KP), and brain natriuretic peptide (BNP), generally referred to herein as “natriuretic peptides,” to patients having Kidney Disease (KD) alone, Heart Failure (HF) alone, KD with concomitant HF, and cardiorenal syndrome (CRS). The continuous ID administration of a natriuretic peptide can be used to maintain in vivo concentrations of the natriuretic peptide above a critical therapeutic efficacy threshold for an extended period of time. However, both acute and chronic delivery, as defined herein, are contemplated for all embodiments of the invention. Both bolus and continuous ID delivery of natriuretic peptides are also contemplated for all embodiments of the invention.

The systems, devices and methods of the invention are also useful for treating other renal or cardiovascular diseases, such as congestive heart failure (CHF), dyspnea, elevated pulmonary capillary wedge pressure, chronic renal insufficiency, acute renal failure, and diabetes mellitus. The medical system and device of the invention can contain an ID drug provisioning component to administer a therapeutically effective amount of natriuretic peptide to a patient suffering from KD alone, HF or with concomitant KD and HF wherein the ID drug provisioning component maintains a plasma concentration of the natriuretic peptide within a specified range. The medical system preferably delivers a natriuretic peptide hormone selected from any one of long-acting natriuretic peptide (LANP), kaliuretic peptide (KP), urodilatin (URO), atrial natriuretic peptide (ANP), brain natriuretic peptide (BNP) and vessel dilator (VD). In any embodiment, the systems, devices and methods of the invention can deliver a chimeric peptide selected from any one of CD-NP (SEQ ID No. 9), CU-NP (SEQ ID No. 10), DNP (SEQ ID No. 8), and CNP (SEQ ID No. 7).

In certain embodiments, an ID drug provisioning component configured with a pumping apparatus is used to administer a therapeutically effective amount of a therapeutic composition from a reservoir. A microneedle array having a substrate with plural microneedles projecting from a surface of the substrate has microneedles, which are in fluid communication with the reservoir. The intra-substrate space can be in fluid communication via a catheter to transport the composition from the reservoir of the drug provisioning component to the intra-substrate space. The device can have a first pressure sensor for sensing a pressure within the catheter and a controller for controlling a pumping rate of the pump and for monitoring a pressure within the catheter to determine if flow through the microneedle array is within an expected range.

In certain embodiments, a medical device has a second pressure sensor for sensing a pressure within an intra-substrate space of a microneedle array having a substrate, wherein the controller monitors a pressure difference between the first pressure sensor and the second pressure sensor and determines if the flow rate between individual microneedles of the microneedle array is substantially equal. In other embodiments, the therapeutic composition can contain one or more natriuretic peptide.

In certain embodiments, a composition having a natriuretic peptide has a concentration of the natriuretic peptide from about 0.1 to about 10 mg/mL.

In some embodiments, microneedles of a microneedle array have a length selected from any of from about 300 to about 1500 μm, from about 500 to about 900 μm, from about 200 to about 1200 μm, from about 300 to about 1000 μm, from about 400 to about 900 μm, from about 600 to about 800 μm and from about 700 to about 900 μm.

In other embodiments, a distal end of a catheter connecting a pump and an intra-substrate space of a microneedle array is divided into plural attachment members, the plural attachments attached to separate ports on the microneedle array.

In any embodiment, a therapeutic composition comprising a natriuretic peptide is administered by intradermal administration to a patient suffering from kidney disease alone, heart failure, concomitant kidney disease and heart failure, or cardiorenal syndrome using a drug provisioning component, and a plasma concentration of the natriuretic peptide is maintained within a specified range, wherein the bioavailability of the natriuretic peptide is increased or the half-life of absorption of the natriuretic peptide is decreased compared to the composition delivered by subcutaneous administration.

A second therapeutic method of treating a patient having KD alone, HF or with concomitant KD and HF, or CRS is provided wherein the method includes increasing plasma or serum concentration of the natriuretic peptide in the patient using the devices and systems of the invention. The method preferably further includes maintaining circulating levels of natriuretic peptide in the plasma or serum of the patient within a specified mean steady state concentration range.

A medical system for administering the natriuretic peptide to a patient having KD alone, HF or with concomitant KD and HF, or CRS is provided. The medical system includes a drug provisioning component that selectively releases a pharmaceutically effective amount of natriuretic peptide to the patient and a control unit having a processor operably connected to and in communication with the drug provisioning component. The control unit is programmed with a set of instructions that causes the drug provisioning component to administer the natriuretic peptide to the patient according to a therapeutic regimen comprising administering a natriuretic peptide to the patient intradermally, wherein the therapeutic regimen is sufficient to maintain circulating levels of the natriuretic peptide in the plasma or serum of the patient above a desired mean steady state concentration. In certain embodiments, the therapeutic regimen is selected to maintain serum natriuretic peptide concentrations in the patient at a value not greater than a critical concentration threshold. In any embodiment of the invention, the natriuretic peptides may include any of the atrial natriuretic peptide (ANP) hormones. These include long acting natriuretic peptide (LANP), kaliuretic peptide (KP), atrial natriuretic peptide (ANP), brain natriuretic peptide (BNP) vessel dilator (VD), and urodilatin (URO). In other embodiments, a chimeric peptide selected from any one of CD-NP (SEQ ID No. 9), CU-NP (SEQ ID No. 10), DNP (SEQ ID No. 8), and CNP (SEQ ID No. 7) can be delivered.

In any embodiment of the invention, the drug provisioning component can deliver the natriuretic peptide at a fixed, pulsed, or variable rate. The drug provisioning component may also be programmable or controllable by the patient.

In any embodiment of the invention, a control unit may operate to regulate the selective release of the natriuretic peptide to maintain a mean steady state concentration using data obtained from the patient. The control unit may further contain computer memory, and the control unit, using the computer memory and processor, may further compile and store a database containing data collected from the patient and also compute a dosing schedule that makes up a part of the therapeutic regimen.

In any embodiment, a method for administering a natriuretic peptide is provided. A natriuretic peptide is administered to a patient suffering from kidney disease alone, heart failure, concomitant kidney disease and heart failure, or cardiorenal syndrome using a drug provisioning component to maintain a plasma level of the natriuretic peptide at a steady state concentration from about 0.5 to about 200 pmol/mL, wherein the natriuretic peptide is administered through an intradermal route. The concentration levels for the natriuretic peptide can also be in the range from 0 to 200 ng/ml, as represented by the range from n to (n+i), where n={xε [symbol] |0<x≦200} and i={yε [symbol] |0≦y≦(200−n)}.

Other objects, features and advantages of the present invention will become apparent to those skilled in the art from the following detailed description. It is to be understood, however, that the detailed description and specific examples, while indicating some embodiments of the present invention are given by way of illustration and not limitation. Many changes and modifications within the scope of the present invention may be made without departing from the spirit thereof, and the invention includes all such modifications.

### BRIEF DESCRIPTION OF THE DRAWINGS

FIG. 1 shows a drug provisioning component with a microneedle array in accordance with some embodiments.

FIG. 2 shows a drug provisioning component with a substrate having a microneedle array in accordance with some embodiments.

FIG. 3 shows a drug provisioning component with a substrate having a microneedle array in accordance with some embodiments.

FIG. 4 shows a substrate having a microneedle array in accordance with some embodiments.

FIGS. 5A and 5B shows plasma concentration data obtained from a 50 μg IV bolus (FIG. 5A) and 50 μg SQ bolus (FIG. 5B).

FIGS. 6A and 6B show a regression line fit for plasma concentration data obtained from a 50 μg IV bolus (FIG. 6A) and 50 μg SQ bolus (FIG. 6B).

FIG. 7 shows a one-compartment model of data obtained from a 50 μg IV bolus (FIG. 7A) and 50 μg SQ bolus (FIG. 7B).

FIGS. 8A (log scale) and 8B (non-log scale) show a simulation of plasma concentration for a 50 μg intradermal bolus of ANP in accordance with a first scenario and FIGS. 8C (log scale) and 8D (non-log scale) show a simulation for a 50 μg intradermal bolus of ANP in accordance with a second scenario. A plot for a 50 μg subcutaneous bolus of ANP is shown in open circles in each of FIGS. 8A-D. The y-axes are in units of pmol/L and the x-axes are in units of minutes.

FIG. 9 shows plots of simulations for infusions of ANP by intradermal delivery in comparison to a plot for subcutaneous infusion of ANP.

FIG. 10 shows two graphs of simulations for infusions of ANP by intradermal delivery with variation in bioavailability.

FIG. 11 shows hypothetical data for stability of a natriuretic peptide in different media.

### DETAILED DESCRIPTION OF THE INVENTION

The invention relates to selective delivery of a natriuretic peptide using a drug provisioning component that employs an array of microneedles to deliver a composition containing the natriuretic peptide to the dermis. A preferred embodiment of the invention contemplates intradermal (ID) delivery using an infusion pump at a continuous rate to maintain a specified plasma concentration of the natriuretic peptides. Natriuretic peptides and their sequences are disclosed in U.S. Pat. No. 5,691,310 and U.S. Patent App. Pub. Nos. 2006/0205642, 2008/0039394, 2009/0062206, and 2009/0170196, each of which is incorporated by reference herein in its entirety.

### DEFINITIONS

Unless defined otherwise, all technical and scientific terms used herein generally have the same meaning as commonly understood by one of ordinary skill in the relevant art. Generally, the nomenclature used herein for drug delivery, pharmacokinetics, pharmacodynamics, and peptide chemistry is well known and commonly employed in the art. Further, the techniques for the discussed procedures are generally performed according to conventional methods in the art.

The articles “a” and “an” are used herein to refer to one or to more than one (i.e., to at least one) of the grammatical object of the article. By way of example, “an element” means one element or more than one element.

The terms “administering,” “administer,” “delivering,” “deliver,” “introducing,” and “introduce” can be used interchangeably to indicate the introduction a compound, agent or peptide into the body of a patient, including methods of introduction where the compound, agent or peptide will be present in the blood or plasma of a subject to whom the compound, agent or peptide is administered.

The term “comprising” includes, but is not limited to, whatever follows the word “comprising.” Thus, use of the term indicates that the listed elements are required or mandatory but that other elements are optional and may or may not be present.

The term “consisting of” includes and is limited to whatever follows the phrase the phrase “consisting of.” Thus, the phrase indicates that the limited elements are required or mandatory and that no other elements may be present.

The phrase “consisting essentially of” includes any elements listed after the phrase and is limited to other elements that do not interfere with or contribute to the activity or action specified in the disclosure for the listed elements. Thus, the phrase indicates that the listed elements are required or mandatory but that other elements are optional and may or may not be present, depending upon whether or not they affect the activity or action of the listed elements.

“Pharmaceutically acceptable” is meant to encompass any carrier, which does not interfere with effectiveness of the biological activity of the active ingredient and that is not toxic to the host to which it is administered.

“Intradermal drug provisioning component,” as used defined herein encompasses any and all devices that administers a therapeutic agent to a subject by intradermal delivery. The drug provisioning component and the control unit may be “co-located,” which means that these two components, in combination, may make up one larger, unified unit of a system.

As used herein, “programmable” refers to a device using computer hardware architecture and being capable of carrying out a set of commands, automatically.

“Glomerular filtration rate” describes the flow rate of filtered fluid through the kidney. The estimated glomerular filtration rate or “eGFR” is a measure of filtered fluid based on a creatinine test and calculating the eGFR based on the results of the creatinine test.

“Intravenous” delivery refers to delivery of an agent by means of a vein.

“Intramuscular” delivery refers to delivery of an agent by means of muscle tissue.

“Subcutaneous” delivery refers to delivery of an agent by means of the subcutis layer of skin directly below the dermis and epidermis.

The term “delivering,” “deliver,” “administering,” and “administers” can be used interchangeably to indicate the introduction of a therapeutic or diagnostic agent into the body of a subject in need thereof to treat a disease or condition, and can further mean the introduction of any agent into the body for any purpose.

The “field of chronic delivery” involves the following four parameters: period of treatment, scope, route of administration, and method of delivery. “Chronic delivery” means a period of treatment or drug delivery of more than 24 hours, even if the drug is not delivered continuously for that period of time. The scope of delivery involves one or more drugs, in any combination. The route of administration includes, but is not limited to, intradermal delivery. The “field of acute delivery” involves the same four parameters as for the field of chronic delivery. The difference between the two fields is the period of treatment. “Acute delivery” means a period of treatment or drug delivery of less than or equal to 24 hours, even if the drug is delivered continuously for that period of time.

“Intradermal delivery” refers to delivery of an agent to the dermis layer of the skin below the epidermis.

“Transdermal delivery” refers to delivery of an agent to the surface of the epidermis immediately below the surface of the epidermis such that the agent can migrate to the circulation.

The term “intradermal space” refers to the extracellular, extravascular volume of the dermis layer of the skin below the epidermis.

The term “therapeutically effective amount” refers to an amount of an agent (e.g., natriuretic peptides) effective to treat at least one symptom of a disease or disorder in a subject. The “therapeutically effective amount” of the agent for administration may vary based upon the desired activity, the diseased state of the subject being treated, the dosage form, method of administration, subject factors such as the subject's sex, genotype, weight and age, the underlying causes of the condition or disease to be treated, the route of administration and bioavailability, the persistence of the administered agent in the body, evidence of natriuresis and/or diuresis, the type of formulation, and the potency of the agent.

The terms “treating” and “treatment” refer to the management and care of a patient having a pathology or condition for which administration of one or more therapeutic compounds or peptides is indicated for the purpose of combating or alleviating symptoms and complications of the condition. Treating includes administering one or more formulations or peptides of the present invention to prevent or alleviate the symptoms or complications or to eliminate the disease, condition, or disorder. As used herein, “treatment” or “therapy” refers to both therapeutic treatment and prophylactic or preventative measures. “Treating” or “treatment” does not require complete alleviation of signs or symptoms, does not require a cure, and includes protocols having only a marginal or incomplete effect on a patient.

The term “therapeutic regimen” is used according to its meaning accepted in the art and refers to, for example, a part of a treatment plan for an individual suffering from a pathological condition that specifies factors such as the agent or agents to be administered to the patient or subject, the doses of such agent(s), the schedule and duration of the treatment, etc.

An “infusion device” or “infusion pump” describes a means for delivering an infusion liquid into a patient or subject. Typically, the infusion pump has three major components: a fluid reservoir, a catheter system for transferring the fluids into the body, and a device that generates and/or regulates flow of the infusion fluid to achieve a desired concentration of a therapeutic agent in the body. One of ordinary skill will appreciate that there are many ways for regulating the flow of the infusion liquid by either mechanical or electric means. Hence, many forms for delivering the liquid are contemplated by the present invention, and such varied embodiments do not depart from the spirit of the invention. For example, the infusion fluid of the invention can be delivered and regulated using either roller pumps or electro-kinetic pumping (also known as electro-osmotic flow). Examples of infusion devices further include, but are not limited to, an external or an implantable drug delivery pumps.

The term “continuous infusion system” refers to a collection of components for continuously administering a fluid to a patient or subject for an extended period of time without having to establish a new site of administration each time the fluid is administered. As in the “infusion device” or “infusion pump,” the fluid in the continuous infusion system typically contains a therapeutic agent or agents. The system typically has one or more reservoir(s) for storing the fluid(s) before the fluid is infused, a pump, and control elements to regulate the pump.

The terms “continuous administration” and “continuous infusion” are used interchangeably herein and mean delivery of an agent, such as an atrial natriuretic peptide, in a manner that, for example, avoids fluctuations in the in vivo concentrations of the agent throughout the course of a treatment period. “Delivery” as used herein, can mean any type of means to effect a result either by electrical, mechanical or other physical means. This can be accomplished by constantly or repeatedly injecting substantially identical amounts of the agent, typically with a continuous infusion pump device, for a set period of time, e.g., at least every hour, 24 hours a day, seven days a week for a period such as at least 3 to 7 days, such that a steady state serum or plasma level is achieved for the duration of the treatment. This can also be described as a cyclic on/off pattern.

A “deliverable amount” is defined as any amount of a measured fluid that can be delivered through a fluid delivery catheter as known by those of ordinary skill in the art. “Delivery” as used herein generally, can mean any type of means to effect a result either by electrical, mechanical or other physical means.

“Risk” relates to the possibility or probability of a particular event occurring either presently or at some point in the future.

The terms “subject” and “patient” can be used interchangeably, and describe a member of any animal species, preferably a mammalian species, optionally a human. The animal species can be a mammal or vertebrate such as a primate, rodent, lagomorph, domestic animal or game animal. Primates include chimpanzees, cynomologous monkeys, spider monkeys, and macaques, e.g., Rhesus or Pan. Rodents and lagomorphs include mice, rats, woodchucks, ferrets, rabbits and hamsters. Domestic and game animals include cows, horses, pigs, sheep, deer, bison, buffalo, mink, felines, e.g., domestic cat, canines, e.g., dog, wolf and fox, avian species, e.g., chicken, turkey, emu and ostrich, and fish, e.g., trout, catfish and salmon. The subject can be an apparently healthy individual, an individual suffering from a disease, or an individual being treated for a disease.

The term “sample” refers to a quantity of a biological substance that is to be tested for the presence or absence of one or more molecules.

“Renin,” also known as angiotensinogenase, is an enzyme that participates in the body's renin-angiotensin system (RAS), which regulates the body's mean arterial blood pressure by mediating extracellular volume (i.e., that of the blood plasma, lymph and interstitial fluid) and arterial vasoconstriction. Renin is released by the kidney when a subject has decreased sodium levels or low blood volume.

“Endogenous” substances are those that originate from within an organism, tissue, or cell.

The term “pharmacokinetics” is used according to its meaning accepted in the art and refers to the study of the action of drugs in the body. Pharmacokinetics includes, for example, the effect and duration of drug action, and the rate at which the drug is absorbed, distributed, metabolized, and eliminated by the body.

The term “pharmacodynamics” is used according to its meaning accepted in the art and refers to the study of the biochemical and physiological effects of drugs on the body, the mechanism of drug action, and the relationship between drug concentration and effect.

The phrase “area under the curve” or “AUC” refers to the area under a plasma concentration versus time curve. It indicates a measurement of drug absorption. AUC is described by the following formula:

AUC=∫₀^{∞}C(t)dt

where C(t) indicates the concentration of the drug in the plasma at time t.

The term “elimination half-life” or “elimination half-time” as generally used herein is the time required for the drug concentration in a compartment (usually the central compartment) from which the drug is being eliminated (by processes such as enzymatic degradation, receptor uptake, glomerular filtration) to decrease by 50%. For first-order elimination processes, the elimination half-life=ln(2)/kₑ, where kₑ is the elimination rate constant. Note that kₑ is equivalent to CL/VOD, where CL is clearance and VOD is volume of distribution.

“Elimination half-life” or “elimination half-time” when used herein in the context of administering a peptide drug to a patient is defined as the time required for the blood plasma concentration of a substance to halve (“plasma half-life”) its concentration in plasma. The knowledge of half-life is useful for the determination of the frequency of administration of a drug for obtaining a desired plasma concentration. Generally, the half-life of a particular drug is independent of the dose administered where first-order kinetic behavior is observed. There could also be more than one half-life associated with the peptide drug depending on multiple clearance mechanisms, redistribution, and other mechanisms known in the art. Usually, alpha and beta half-lives are defined such that the alpha phase is associated with redistribution, and the beta phase is associated with clearance. For protein drugs that are, for the most part, confined to the bloodstream, there can be at least two clearance half-lives.

The term “terminal half-live” refers to the time required for the concentration of a drug in the sampled compartment (usually the central compartment or the blood stream) to decrease by 50%. Terminal half-life can be equal to the elimination or absorption half-life. Generally, the terminal half life is equal to [ln (2)]/λ_{z}, where λ_{z} is the slope of the log(Cp) versus time curve (or data).

The term “mean residence time” refers to the time required for the amount of drug in the body to decrease by 50% after administration or the average residence time of drug molecule in the body.

The term “absorption half-life” or “absorption half-time” refers to the time required for 50% of drug administered to the extravascular space to be (or appear to be) absorbed into the vasculature or central compartment. For first-order absorption, the absorption half-life=ln(2)/kₐ, where kₐ is the absorption rate constant. In some systems (or models), two rate constants contribute to the observed absorption half-life. First, a rate constant for movement of the drug from a site of administration (extravascular space) to a central compartment (kₐ₁), and second, a rate constant for movement of the drug from the extravascular by means of elimination or another pathway that makes the drug unavailable for movement into the central compartment (kₐ₂).

“Elimination” refers to the removal or transformation of a drug in circulation, usually via the kidney and liver, or by enzymes or cellular receptors.

“Absorption” refers to the transition of drug from the site of administration to the blood circulation.

The term “specified range,” as used herein contemplates both a measured value, such as the concentration value of an agent or peptide in the plasma of a patient, and a measured value that is either added or subtracted from a normal or basal level of a subject.

“Loading dose” refers to the dose(s) of drugs given at the onset of therapy to rapidly provide a therapeutic effect. Use of a loading dose prior to a maintenance dosage regimen will shorten the time required to approach a steady state.

In pharmacokinetics, “steady state” represents the equilibrium between the amount of drug given and the amount eliminated over the dosing interval. In general, it takes drug four to five half-lives to reach a steady state, however the multiple can vary depending on the route of administration. Sampling should occur when the drug has reached its steady state to judge efficacy and toxicity of the drug therapy. Steady state should not be confused with the therapeutic range.

“Mean steady state concentration,” denoted by “Css” refers to the concentration of a drug or chemical in a body fluid, usually plasma, at the time a “steady state” has been achieved and rates of drug administration and drug elimination are equal. Steady state concentrations fluctuate between a maximum (peak) (“Cmax”) and minimum (trough) (“Cmin”) concentration with each dosing interval. Css is a value approached as a limit and is achieved following the last of an infinite number of equal doses given at equal intervals.

“Plasma concentration” (Cp) refers to the amount of a drug in the blood plasma of the patient.

“Maximum plasma concentration” (Cₘₐₓ) refers to the maximum amount of a drug observed in the blood of a patient or subject.

“Average plasma concentration” (C_{avg}) refers to the average amount of a drug observed in the blood of a patient or subject over a time course of a period of observation of the amount of the drug in the blood.

“Minimum plasma concentration” (Cₘᵢₙ) refers to the minimum amount of a drug observed in the blood of a patient or subject over a time course of a period of observation of the amount of the drug in the blood.

“Time to maximum concentration” (Tₘₐₓ) refers to the time observed to reach maximum plasma concentration of a drug as measured from the initiation of regimen of administration of the drug.

“Percent fluctuation” (% Fluctuation) refers to the difference between Cₘₐₓ and Cₘᵢₙ for a drug in the blood over a time course of a period of observation of the amount of the drug in the blood, where

$$\% Fluctuation = \frac{{C}_{max} - {C}_{min}}{{C}_{avg}} \times 100.$$

The “volume of distribution” (VOD) is a hypothetical volume that is the proportionality constant which relates the concentration of drug in the blood or serum and the amount of drug in the body.

“Pharmacokinetic constraints,” as used herein, describes any factor that determines the pharmacokinetic profile of a drug such as immunogenicity, route of administration, endogenous concentrations of the natriuretic peptides, diurnal variation, and rate of drug delivery.

A “dose-response” relationship describes how the likelihood and severity of adverse health effects (i.e., the responses) are related to the amount and condition of exposure to an agent (i.e., the dose provided). Dose-response assessment is a two step process. The first step involves an assessment of all data that are available or can be gathered through experiments, in order to document the dose-response relationship(s) over the range of observed doses (i.e., the doses that are reported in the data collected). However, frequently this range of observation may not include sufficient data to identify a dose where the adverse effect is not observed (i.e., the dose that is low enough to prevent the effect) in the human population. The second step consists of extrapolation to estimate the risk, or probably of adverse effect, beyond the lower range of available observed data to make inferences about the critical region where the dose level begins to cause the adverse effect in the test population.

“Selective release” of an atrial natriuretic peptide as used in the invention describes the controlled delivery of a therapeutic using the ID drug delivery component, and can also refer to a controlled or programmed release of the atrial natriuretic peptide into the vasculature of the patient, according to a treatment protocol, through use of the drug provisioning component.

The term “distal tip” or “distal end” refers to the end that is situated farthest from a point of attachment or origin, and the end closest to the point of attachment or origin is known as the “proximal” end.

The term “peptide,” as used herein, describes an oligopeptide, polypeptide, peptide, protein or glycoprotein, and includes a peptide having a sugar molecule attached thereto. As used herein, “native form” means the form of the peptide when produced by the cells and/or organisms in which it is found in nature. When the peptide is produced by a plurality of cells and/or organisms, the peptide may have a variety of native forms. “Peptide” can further refer to a polymer in which the monomers are amino acids that are joined together through amide bonds. Also included are peptides which have been modified using ordinary molecular biological techniques so as to improve their resistance to proteolytic degradation or to optimize solubility properties or to render them more suitable as a therapeutic agent. Analogs of such peptides include those containing residues other than naturally occurring L-amino acids, e.g., D-amino acids or non-naturally occurring synthetic amino acids. The present invention also embraces recombination peptides such as recombinant human ANP (hANP) obtained from bacterial cells after expression inside the cells. It will be understood by those of skill in the art that the peptides and recombinant peptides of the present invention can be made by varied methods of manufacture wherein the peptides of the invention are not limited to the products of any of the specific exemplary processes listed herein.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. The present invention also provides for analogs of proteins or peptides which comprise a protein as identified above.

The term “fragment,” as used herein, refers to a polypeptide that comprises at least six contiguous amino acids of a polypeptide from which the fragment is derived. In preferred embodiments, a fragment refers to a polypeptide that comprises at least 10 contiguous amino acids of a polypeptide from which the fragment is derived, more preferably at least 10 contiguous amino acids, still more preferably at least 15 contiguous amino acids, and still more preferably at least 20 contiguous amino acids of a polypeptide from which the fragment is derived.

The term “natriuretic peptide fragment” refers to a fragment of any natriuretic peptide defined and described herein.

As used herein, “cardiovascular disease” refers to various clinical diseases, disorders or conditions involving the heart, blood vessels, or circulation. Cardiovascular disease includes, but is not limited to, coronary artery disease, peripheral vascular disease, hypertension, myocardial infarction, and heart failure.

The terms “natriuretic” or “natriuresis” refer to the ability of a substance to increase sodium clearance from a subject.

The terms “renal or cardiovascular protective” and “renal or cardiovascular protective effects” refer to the ability of a substance to improve one or more functions of the kidneys or heart of a subject, including natriuresis, diuresis, cardiac output, hemodynamics, renal cortical blood flow or glomerular filtration rate, or to lower the blood pressure of the subject. Any measurable diagnostic factor that would be recognized by one having skill in the art as reducing stress on the kidneys and/or heart or as evidence of improvement in the function of the renal or cardiovascular system can be considered a renal or cardiovascular protective effect. The term “renal protective” or “renal protective effect” refers to a measurable diagnostic factor that would be recognized by one having skill in the art as particularly related to an indication of reduced stress on the kidneys or improvement in renal function. The term “cardiovascular protective” or “cardiovascular protective effect” refers to a measurable diagnostic factor that would be recognized by one having skill in the art as particularly related to an indication of reduced stress on the cardiovascular system or improvement in cardiac function.

As used herein, “heart failure” (HF) refers to a condition in which the heart cannot pump blood efficiently to the rest of the body. Heart failure may be caused by damage to the heart or narrowing of the arteries due to infarction, cardiomyopathy, hypertension, coronary artery disease, valve disease, birth defects or infection. Heart failure may also be further described as chronic, congestive, acute, decompensated, acute decompensated, systolic, or diastolic. The NYHA classification describes the severity of the disease based on functional capacity of the patient and is incorporated herein by reference. Heart failure can be with preserved ejection fraction or be with reduced ejection fraction. Further, heart failure can include left heart failure or right heart failure.

The “renal system,” as defined herein, comprises all the organs involved in the formation and release of urine including the kidneys, ureters, bladder and urethra.

“Kidney disease” (KD) is a condition characterized by the slow loss of kidney function over time. The most common causes of KD are high blood pressure, diabetes, heart disease, and diseases that cause inflammation in the kidneys. Kidney disease can also be caused by infections or urinary blockages. If KD progresses, it can lead to end-stage renal disease (ESRD), where the kidneys fail completely. In the Cardiorenal Syndrome (CRS) classification system, CRS Type I (Acute Cardiorenal Syndrome) is defined as an abrupt worsening of cardiac function leading to acute kidney injury; CRS Type II (Chronic Cardiorenal syndrome) is defined as chronic abnormalities in cardiac function (e.g., chronic congestive heart failure) causing progressive and permanent kidney disease; CRS Type III (Acute Renocardiac Syndrome) is defined as an abrupt worsening of renal function (e.g., acute kidney ischaemia or glomerulonephritis) causing acute cardiac disorders (e.g., heart failure, arrhythmia, ischemia); CRS Type IV (Chronic Renocardiac syndrome) is defined as kidney disease (e.g., chronic glomerular disease) contributing to decreased cardiac function, cardiac hypertrophy and/or increased risk of adverse cardiovascular events; and CRS Type V (Secondary Cardiorenal Syndrome) is defined as a systemic condition (e.g., diabetes mellitus, sepsis) causing both cardiac and renal dysfunction (Ronco et al., Cardiorenal syndrome, J. Am. Coll. Cardiol. 2008; 52:1527-39). KD can be referred to by different stages indicated by Stages 1 to 5. Stage of KD can be evaluated by glomerular filtration rate of the renal system. Stage 1 KD can be indicated by a GFR greater than 90 mL/min/1.73 m² with the presence of pathological abnormalities or markers of kidney damage. Stage 2 KD can be indicated by a GFR from 60-89 mL/min/1.73 m², Stage 3 KD can be indicated by a GFR from 30-59 mL/min/1.73 m² and Stage 4 KD can be indicated by a GFR from 15-29 mL/min/1.73 m². A GFR less than 15 mL/min/1.73 m² indicates Stage 5 KD or ESRD. It is understood that KD, as defined in the present invention, contemplates KD regardless of the direction of the pathophysiological mechanisms causing KD and includes CRS Type II and Type IV and Stage 1 through Stage 5 KD among others. Kidney disease can further include acute renal failure, acute kidney injury, and worsening of renal function.

A “control system” consists of combinations of components that act together to maintain a system to a desired set of performance specifications. The performance specifications can include sensors and monitoring components, processors, memory and computer components configured to interoperate.

A “controller” or “control unit” is a device which monitors and affects the operational conditions of a given system. The operational conditions are typically referred to as output variables of the system, which can be affected by adjusting certain input variables.

By the phrase, “in communication,” it is meant that the elements of the system of the invention are so connected, either directly or remotely, wirelessly or by direct electrical contact so that data and instructions can be communicated among and between said elements.

“Controlled delivery” refers to the implementation of a controller or control unit that is either programmable or patient-controlled that causes the drug delivery component to administer the therapeutic agent to the patient according to a programmed administration protocol or in response to a command given by the patient or a healthcare provider.

“Patient controlled” delivery refers to mechanisms by which the patient can administer and/or control the administration of a drug. Thus, the patient can cause the drug delivery component to administer the therapeutic formulation.

The term “a cyclic on/off pattern” as used herein means a repetitive condition which alternates between being in “on” and “off” states. Such conditions may pertain to drug delivery by a drug provisioning component of a medical system wherein the “on” state denotes a period of drug delivery. A drug administered in “a cyclic on/off pattern” is delivered as repetitive doses over duration of time.

The term “maintaining a plasma concentration” refers to, in some embodiments, maintaining a concentration of a compound or peptide in the plasma of a subject at a recited or referenced concentration range by administration of the compound or peptide by any appropriate means. In certain other embodiments, “maintaining a plasma concentration” refers to maintaining a concentration of a compound or peptide at a concentration in the plasma of a subject that is in addition to an endogenous concentration of that compound or peptide. Where the compound or peptide is a naturally occurring substance, a subject can have an endogenous baseline of that compound or peptide measurable in the plasma. Maintaining a plasma concentration at a recited concentration can refer to increasing the plasma concentration of the compound or peptide by the recited amount and maintaining a plasma concentration at that elevated amount.

The term “multiple days” refers to any duration of time greater than 24 hours.

The term “pulmonary capillary wedge pressure” refers to the pressure measured by wedging a pulmonary catheter with a deflated balloon into a small pulmonary arterial branch.

Measurements of pharmacokinetic variables such as steady state concentration, absorption half-life, administration rate, volume of distribution, elimination half-life, and clearance are described as ranges. The measurement ranges are represented by equations encompassing groups of ranges. Specifically, the values of pharmacokinetic variables are described as ranges from n to (n+i), wherein the definitions of n and i are specific to a particular pharmacokinetic variable. It is to be understood that a given range supports every possible permutation thereof, and accordingly all such permutations are therefore contemplated by the invention.

As used herein, a range from n to (n+i) is subject to the constraints n={xεR|α≦x≦β}, for α≠0, and i={yεR|0≦y≦(β−n)}, or n={xεR|α<x≦β} for α≧0, and i={yεR|0≦y≦(β−n)}, or other similar constraints, where α is a minimum value specific to a pharmacokinetic variable, and β is a maximum value specific to a pharmacokinetic variable. Such a range, n to (n+i), also inherently supports any sub-range falling within the larger range.

In an example where α=0, and β=500, a range from n to (n+i) where n={xεR|0<x≦500}, and i={yεR|0≦y≦(500−n)}, would encompass all values ranging from greater than 0 up to and including 500, and additionally all sub-ranges within the range of 0 to 500. Specifically, for this example range, a lower bound may be chosen such that x=0.5 meaning the lower bound, n, of a sub-range is 0.5, and the upper bound, (n+i), could be any value from 0.5 to 500. Any sub-range lower bound may be chosen subject to the constraints. For example, if x=10, the lower bound of the sub-range would be 10, and the upper bound could be any value from 10 to 500, thus yielding sub-ranges such as 10-10, 10-10.5, 10-20, 10-25.6, . . . , 10-500. Likewise, if x=45.3, the lower bound of the sub-range would be 45.3, and the upper bound could be any value from 45.3 to 500, thus yielding sub-ranges such as 45.3-45.3, 45.3-45.4, 45.3-46.5, . . . , 45.3-500.

In an example where α=2, and β=450, a range from n to (n+i) where n={xεR|2<x≦450}, and i={yεR|0≦y≦(450−n)}, would encompass all values ranging from greater than 2 up to and including 450, and additionally all sub-ranges within the range of 2 to 450. Specifically, for this example range, a lower bound may be chosen such that x=2.5 meaning the lower bound, n, of a sub-range is 2.5, and the upper bound, (n+i), could be any value from 2.5 to 450. Any sub-range lower bound may be chosen subject to the constraints. For example, if x=10, the lower bound of the sub-range would be 10, and the upper bound could be any value from 10 to 450, thus yielding sub-ranges such as 10-10, 10-10.5, 10-20, 10-25.6, . . . , 10-450. Likewise, if x=45.3, the lower bound of the sub-range would be 45.3, and the upper bound could be any value from 45.3 to 450, thus yielding sub-ranges such as 45.3-45.3, 45.3-45.4, 45.3-46.5, . . . , 45.3-450.

In an example where α=2, and β=450, a range from n to (n+i) where n={xεR|2≦x≦450}, and i={yεR|0≦y≦(450−n)}, would encompass all values ranging from 2 up to and including 450, and additionally all sub-ranges within the range of 2 to 450. Specifically, for this example range, a lower bound may be chosen such that x=2 meaning the lower bound, n, of a sub-range is 2, and the upper bound, (n+i), could be any value from 2 to 450. Any sub-range lower bound may be chosen subject to the constraints. For example, if x=10, the lower bound of the sub-range would be 10, and the upper bound could be any value from 10 to 450, thus yielding sub-ranges such as 10-10, 10-10.5, 10-20, 10-25.6, . . . , 10-450. Likewise, if x=45.3, the lower bound of the sub-range would be 45.3, and the upper bound could be any value from 45.3 to 450, thus yielding sub-ranges such as 45.3-45.3, 45.3-45.4, 45.3-46.5, . . . , 45.3-450. Accordingly, all permutations of a broad range and a sub-range therein are contemplated by the range equations described.

Rates of administration of a natriuretic peptide or other material can be expressed as an absolute rate of a weight or mole amount of the peptide per unit of time or as a weight-based rate that varies based on a subject's weight. For example, the term 10 ng/kg·min means that 10 ng of a natriuretic peptide is administered to the subject every minute for every kg of body weight of the subject. As such, an 85-kg subject receiving a weight-based dose of 10 ng/kg·min receives 850 ng/min of the natriuretic peptide or an absolute rate of 51 μg/hr of the natriuretic peptide. The units ng/kg·min, ng/(kg·min), ng kg⁻¹ min⁻¹ and ng/kg/min are equivalent and have the same meaning as described herein. All mass values can also be expressed in mole terms, for example pmol, with the same meaning as described above.

### Natriuretic Peptides

Natriuretic peptides are a family of peptides acting in the body to oppose the activity of the renin-angiotensin system. In humans, the family consists of atrial natriuretic peptide (ANP) of myocardial cell origin, C-type natriuretic peptide (CNP) of endothelial origin, brain natriuretic peptide (BNP) and urodilatin (URO), which is thought to be derived from the kidney. Atrial natriuretic peptide (ANP), alternatively referred to in the art as atrial natriuretic factor (ANF), is secreted by atrial myocytes in response to increased intravascular volume. Once ANP is in the circulation, its effects are primarily on the kidney, vascular tissue, and adrenal gland. ANP leads to the excretion of sodium and water by the kidneys and to a decrease in intravascular volume and blood pressure. Brain natriuretic peptide (BNP) also originates from myocardial cells and circulates in human plasma similar to ANP. BNP is natriuretic, renin inhibiting, vasodilating, and lusitropic. C-type natriuretic peptide (CNP) is of endothelial cell origin and functions as a vasodilating and growth-inhibiting polypeptide.

The five major ANP hormones are atrial long-acting natriuretic peptide (LANP), kaliuretic peptide (KP), urodilatin (URO), atrial natriuretic peptide (ANP), and vessel dilator (VD). These hormones function via well-characterized particulate guanylyl cyclase receptors linked to cGMP, and have significant blood pressure lowering, diuretic, sodium and/or potassium excreting properties in healthy humans. The peptide sequences for these four ANP peptide hormones are as follows:

proANP or LANP, (a.a. 1-30)
(SEQ ID No. 1)
NPMYNAVSNADLMDFKNLLDHLEEKMPLED
Vessel Dilator, (a.a. 31-67)
(SEQ ID No. 2)
EVVPPQVLSEPNEEAGAALSPLPEVPPWTGEVSPAQR
Kaliuretic Peptide, (a.a. 79-98)
(SEQ ID No. 3)
SSDRSALLKSKLRALLTAPR
ANP, (a.a. 99-126)
(SEQ ID No. 4)
SLRRSSCFGGRMDRIGAQSGLGCNSFRY

The fifth member of the atrial natriuretic peptide family, urodilatin (URO) (ANP a.a. 95-126) is isolated from human urine and has an N-terminal extension of four additional amino acids, as compared with the circulating form of ANP (a.a. 99-126). The sequence for urodilatin is provided in SEQ ID No. 5.

Urodilatin (a.a. 95-126)
(SEQ ID No. 5)
TAPRSLRRSSCFGGRMDRIGAQSGLGCNSFRY

The peptide sequence for BNP is as follows:

BNP
(SEQ ID No. 6)
SPKMVQGSGCFGRKMDRISSSSGLGCKVLRRH

Two chimeric natriuretic peptides are also contemplated by the invention. The first of these is known as CD-NP (SEQ ID No. 9), which comprises the 22 amino acid human C-type natriuretic peptide (CNP), described as (SEQ ID No. 7), and the 15 amino acid C-terminus of Dendroaspis natriuretic peptide (DNP) (SEQ ID No. 8) as described in U.S. Pat. No. 7,754,852, the contents of which are incorporated in their entirety by reference. CD-NP is designed to enhance the renal actions of CNP, which is a ligand for natriuretic peptide receptor B (NPR-B), without inducing excessive hypotension.

CNP
(SEQ ID No. 7)
GLSKGCFGLKLDRIGSMSGLGC
CD-NP
(SEQ ID No. 9)
GLSKGCFGLKLDRIGSMSGLGCPSLRDPRPNAPSTSA
DNP (C-terminus)
(SEQ ID No. 8)
PSLRDPRPNAPSTSA

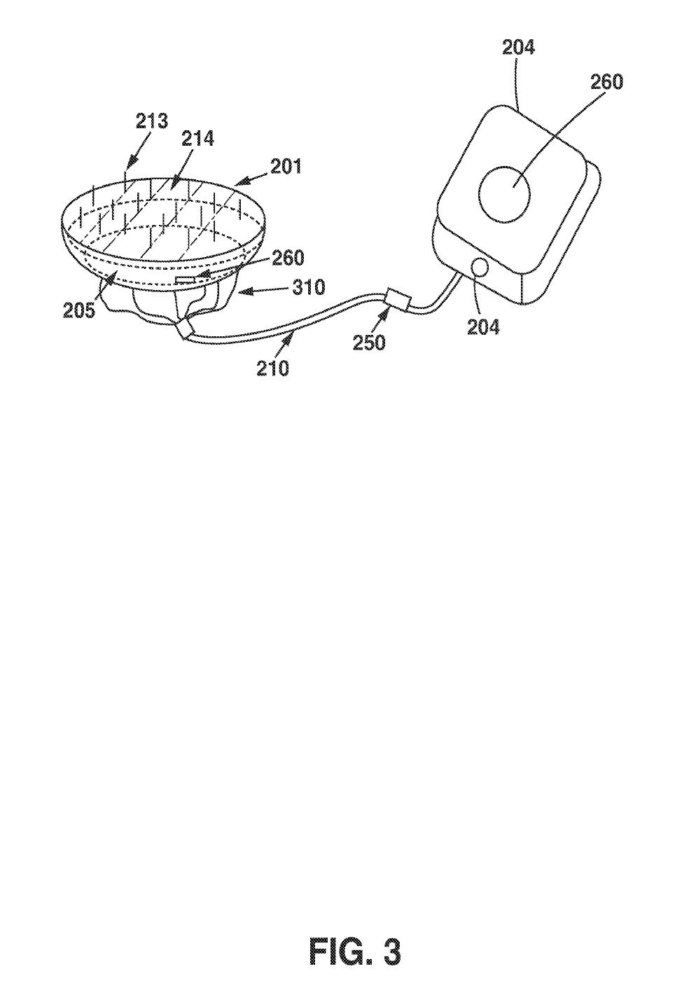

Similarly, the chimeric natriuretic peptide CU-NP (SEQ ID No. 10) is designed to preserve the favorable actions of urodilatin (URO), which is a natriuretic peptide receptor A (NPR-A) agonist, while also minimizing hypotension. CU-NP consists of the 17 amino acid ring of human CNP (SEQ ID No. 5) and the N- and C-termini of urodilatin (SEQ ID Nos. 12 and 13, respectively). FIG. 3 is a schematic diagram of the CU-NP polypeptide (SEQ ID No. 16) that is 32 amino acid residues in length. The first ten amino acid residues of CU-NP (SEQ ID No. 10) correspond to amino acid residues 1 to 10 of urodilatin (SEQ ID No. 12). Amino acid residues 11 to 27 of CU-NP correspond to amino acid residues 6 to 22 of human mature CNP (SEQ ID No. 11). Amino acid residues 28 to 32 of CU-NP correspond to amino acid residues 26 to 30 of Urodilatin (SEQ ID No. 13).

CU-NP
(SEQ ID No. 10)
TAPRSLRRSSCFGLKLDRIGSMSGLGCNSFRY
(SEQ ID No. 11)
CFGLKLDRIGSMSGLGC
(SEQ ID No. 12)
TAPRSLRRSS
(SEQ ID No. 13)
NSFRY

A variant of CD-NP is a peptide having the sequence GLSKGCFGRKMDRIGSMSGLGCPSLRDPRPNAPSTSA (SEQ ID No. 14), which differs in amino acid residues 9-11 compared with CD-NP peptide (SEQ ID No. 9) and has the two cysteine residues involved in a disulfide bond. SEQ ID No. 14, which can be referred to as B-CDNP, has a higher affinity for binding NPR-A and produces higher guanylyl cyclase activity in NPR-A compared with CD-NP peptide. B-CDNP peptide retains the ability to activate NPR-B as well.

An additional variant of CD-NP is a peptide having the sequence GLSKGCFGLKLDRISSSSGLGCPSLRDPRPNAPSTSA (SEQ ID No. 15), which differs in amino acid residues 15-17 compared with CD-NP peptide (SEQ ID No. 9) and has the two cysteine residues involved in a disulfide bond. SEQ ID No. 15, which can be referred to as CDNP-B, has the ability to act as a full agonist for NPR-A in a manner similar to BNP while maintaining an ability to activate NPR-B as well.

Natriuretic peptides as defined herein expressly include variants of CD-NP (SEQ ID No. 9), B-CDNP (SEQ ID No. 14) and CDNP-B (SEQ ID No. 15) having an ability to activate NPR-A and/or NPR-B, where no more than 1, no more than 2, no more than 3, no more than 4, or no more than 5 amino acid residues of the sequences are added, deleted or substituted. Variants include peptides where there is a combination of additions, deletions or substitutions. Substitution of amino acid residues refers to the replacement of any amino acid residue of SEQ ID No.'s 9, 14 and 15 with any other amino acid residue. Further, amino acid substitutions can be conservative amino acid substitutions. Conservative amino acid substitutions are substitutions where an amino acid residue is replaced with another amino acid residue having similar, size, charge, hydrophobicity and/or chemical functionality. Non-limiting examples of conservative amino acid substitutions include, but are not limited to, replacing an amino acid residue appearing in one of the following groups with another amino acid residue from the same group: 1) aspartic acid and glutamic acid as acidic amino acids; 2) lysine, arginine, and histidine as basic amino acids; 3) leucine, isoleucine, methionine, valine and alanine as hydrophobic amino acids; 4) serine, glycine, alanine and threonine as hydrophilic amino acids; 5) glycine, alanine, valine, leucine, isoleucine as aliphatic group residues; 6) a group of amino acids having aliphatic-hydroxyl side chains including serine and threonine; 7) a group of amino acids having amide-containing side chains including asparagine and glutamine; 8) a group of amino acids having aromatic side chains including phenylalanine, tyrosine, and tryptophan; 9) a group of amino acids having basic side chains including lysine, arginine, and histidine; and 10) a group of amino acids having sulfur-containing side chains including cysteine and methionine. The ability of variants to activate NPR-A or NPR-B can be assessed using the assays described in International Patent Publication WO 2010/048308 (PCT/US2009/061511), which is incorporate herein by reference.

In certain embodiments, a variant of CD-NP (SEQ ID No. 9), B-CDNP (SEQ ID No. 14) or CDNP-B (SEQ ID No. 15) has less than about 42 amino acid residues. Variants of B-CDNP peptide expressly includes variants having the sequence GLSKGCFGX₁X₁X₂DRIGSMSGLGCPSLRDPRPNAPSTSA (SEQ ID No. 16) and variants of CDNP-B peptide include GLSKGCFGLKLDRIX₃X₃X₃SGLGCPSLRDPRPNAPSTSA (SEQ ID No. 17), wherein X₁ is selected from the group consisting of lysine, arginine, and histidine, X₂ is selected from the group consisting of leucine, isoleucine, methionine, valine and alanine, and X₃ is selected from the group consisting of serine, glycine, alanine and threonine. CD-NP is currently under clinical study.

One peptide-based pharmaceutical approach to treat HF and/or KD is the use of Nesiritide (B-type natriuretic peptide), which is an FDA approved therapeutic option that lowers elevated filing pressures and improves dyspnea. Nesiritide is the recombinant form of the 32 amino acid human B-type natriuretic peptide, which is normally produced by the ventricular myocardium. The drug facilitates cardiovascular fluid homeostasis through counter-regulation of the renin-angiotensin-aldosterone system and promotion of vasodilation, natriuresis, and diuresis. Nesiritide is administered intravenously usually by bolus injection, followed by IV infusion. Another approved atrial natriuretic type peptide is human recombinant atrial natriuretic peptide (ANP), Carperitide, which has been approved for the clinical management of ADHF in Japan since 1995. Carperitide is also administered via intravenous infusion. Another peptide under study is human recombinant urodilatin (URO), Ularitide.

### Drug Delivery of Natriuretic Peptides

Plasma levels of natriuretic peptides can be increased by causing the selective release of natriuretic peptide using an intradermal (ID) drug provisioning component via ID delivery of a composition containing one or more natriuretic peptide. A control unit may also be present that is connected to and in communication with the drug provisioning component. The control unit of the invention contains a set of instructions that causes the drug provisioning component to administer the natriuretic peptide to the patient according to a therapeutic regimen.

Improvements in bioavailability and absorption may be achieved by varying the manner of administration. Without being limited to any particular theory, intradermal delivery to the highly vascularized dermis may increase access in the lymph capillaries that may be more adept in absorbing peptide molecules. Harvey et al. reports high bioavailability for Entanercept® and Somatropin®. Harvey et al., Pharm. Res. 28:107-26 (2010). As such, the bioavailability of protein drugs may not always be improved. See Gupta et al., Diabetes Tech. & Therapeutics 13:451-56 (2011) (reporting an changed absorption half-life by intradermal delivery compared to subcutaneous delivery with no significant change in AUC and bioavailability) and Pettis et al., Diabetes Tech. & Therapeutics 13:435-42 (2011) (reporting an increased absorption rate of insulin by intradermal delivery with a concurrent inability to enhance bioavailability of insulin). The non-predictable effects on bioavailability and absorption that can be displayed by different modes of administration complicate identifying a most-preferred route of administration. That is, a sharp increase in absorption is not always desirable if a gradual build-up of a drug in the plasma is desired while an attendant improvement in bioavailability may result in cost savings by economizing the use of expensive biologic drugs. Further, the site of administration on the body can add further complexity to observed pharmacokinetic behavior.

A specialized drug provisioning component is configured for intradermal delivery of peptides and other biologic drugs to ensure that the peptides are delivered to a proper depth that is shallower than the depth characterized by subcutaneous delivery. The microneedles are hollow to a sufficient diameter to allow for the liquid composition to pass through the lumen of the microneedles. The microneedles are typically connected to a backing or substrate that allows for the microneedles to penetrate the skin by a specific depth to deliver the composition to the dermis. That is, the microneedles have dimensions to penetrate the stratum corneum such that distal ends of the microneedles allow access to the vascularized dermis and not the denser areas underlying the dermis region of the skin.

In some embodiments, the microneedles have a length from about 300 to about 1500 μm. In other embodiments, the microneedles have a length from about 500 to about 900 μm. In still other embodiments, the microneedles have a length from any of from about 200 to about 1200 μm, from about 300 to about 1000 μm, from about 400 to about 900 μm, from about 600 to about 800 μm and from about 700 to about 900 μm. The length of the microneedles is the length of the microneedles extending from a backing such that the distance of penetration into the skin is controlled.

The microneedles can be arranged in any geometric pattern and can be evenly or irregularly spaced. In some embodiments, an array contains from about 8 to about 100 microneedles. In other embodiments, an array contains from about 10 to about 50 microneedles. In still other embodiments, an array contains any from about 12 to about 80 microneedles, from about 18 to about 70 microneedles, from about 18 to about 50 microneedles and from about 18 to about 40 microneedles. In some embodiments, the microneedles are arranged over an area from about 0.1 to about 20 cm². In other embodiments, the microneedles are arranged over an area from about 0.5 to about 5 cm². In still other embodiments, the microneedles are arranged over an area from any of about 0.5 to about 10 cm², from 1 to about 10 cm², from about 0.5 to about 3 cm² and from about 0.5 to about 2 cm².

The stratum corneum is a layer of dead cells forming the outer boundary of the epidermis. The stratum corneum has a high concentration of keratin that forms an effective boundary to most proteins and peptides. Underneath the epidermis is the dermis region of the skin, which contains a significant amount of blood vessels. The dermis is the closest vascularized region of the body to the surface of the skin. Delivery to the dermis, or intradermal delivery, can provide a convenient route for the delivery of protein- or peptide-based drugs. Delivery of peptide-based pharmaceuticals to the dermis region (intradermally) can result in quicker absorption, improved bioavailability and improved pharmacokinetics compared with subcutaneous delivery.

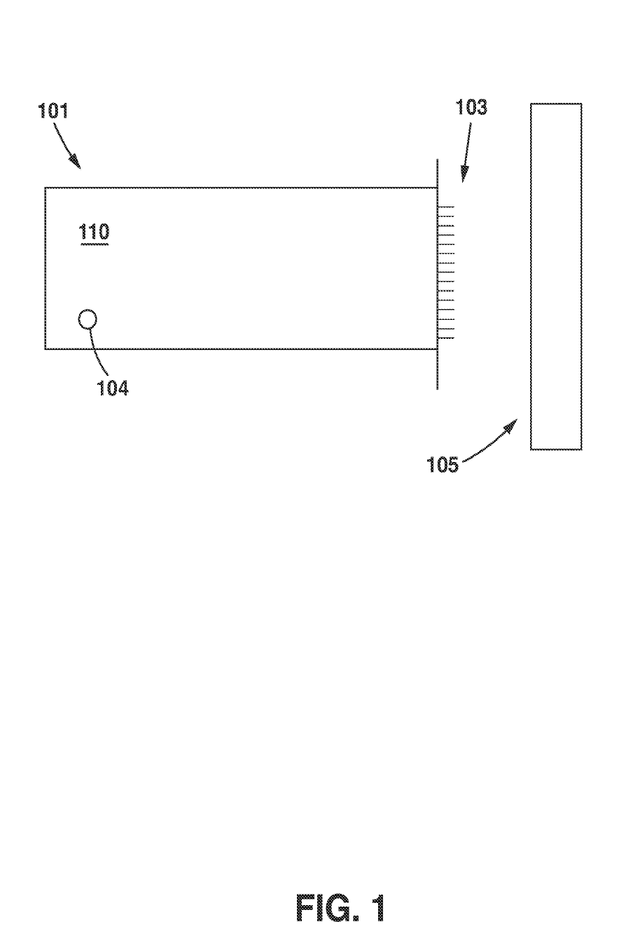

FIG. 1 illustrates an exemplary drug delivery component or drug delivery pump 101 with a microneedle array 103 that penetrates the skin 105 of a patient. The microneedle array 103 is attached to a housing 110. In some embodiments, the housing 110 has a reservoir that can be refilled through a hole or port 104. The microneedles have a relatively small internal diameter as dictated by their small lengths and need for painless entry into the skin. Due to the viscosity of aqueous compositions, a required amount of force is necessary to force fluid from the reservoir through the microneedle array 103. In some embodiments, the housing 110 has a piston for applying a sufficient amount of force to the fluid composition contained in the reservoir to drive delivery through the microneedle array 103. In other embodiments, another suitable mechanism for applying a sufficient force to the fluid composition to affect delivery through the microneedle array is present such as a displacement pump or any suitable means to apply a force or suction. The microneedle array 103 can be retractable or spring loaded to provide for energy stored in one or more springs to provide the necessary force for the microneedle array 103 to penetrate the skin 105.

In other embodiments, the drug provisioning component is configured to impact the basal rate of infusion of the therapeutic formulation by ID administration. The “basal rate” is the continuous infusion rate of the drug that can be programmed. The drug provisioning component can be an infusion apparatus designed to implement a bolus infusion rate. “Bolus” infusion is a rapid infusion of a drug to expedite the effect rapidly by increasing drug concentration level in the blood. The drug provisioning component can be configured to use both basal rate and bolus rate infusion or to use only one infusion method, either basal rate or bolus. The drug provisioning component may also be configured to deliver a drug in a cyclic on/off or repeating pattern alternating between an “on” and “off” state where the drug is delivered as a set of repetitive doses over duration of time.

In some embodiments, the rate of delivery through the microneedle array is from about 10 to about 200 μL/min. In other embodiments, the rate of delivery through the microneedle array is from about 5 to about 150 μL/min. In further embodiments, the rate of delivery through the microneedle array is any from about 3 to about 100 μL/min, from about 1 to about 50 μL/min, from about 1 to about 75 μL/min, from about 1 to about 20 μL/min, from about 1 to about 15 μL/min and from about 1 to about 10 μL/min.

In some embodiments, the drug provisioning component is adapted for chronic delivery of a composition containing a natriuretic peptide. For chronic delivery, the microarray is engaged with a patient for an extended period of time that can be greater than 24 hours up to a period that can span weeks or more. During the period of chronic administration, the composition with the natriuretic peptide can be delivered by continuous infusion, by an intermittent bolus or a combination of continuous infusion and intermittent bolus.

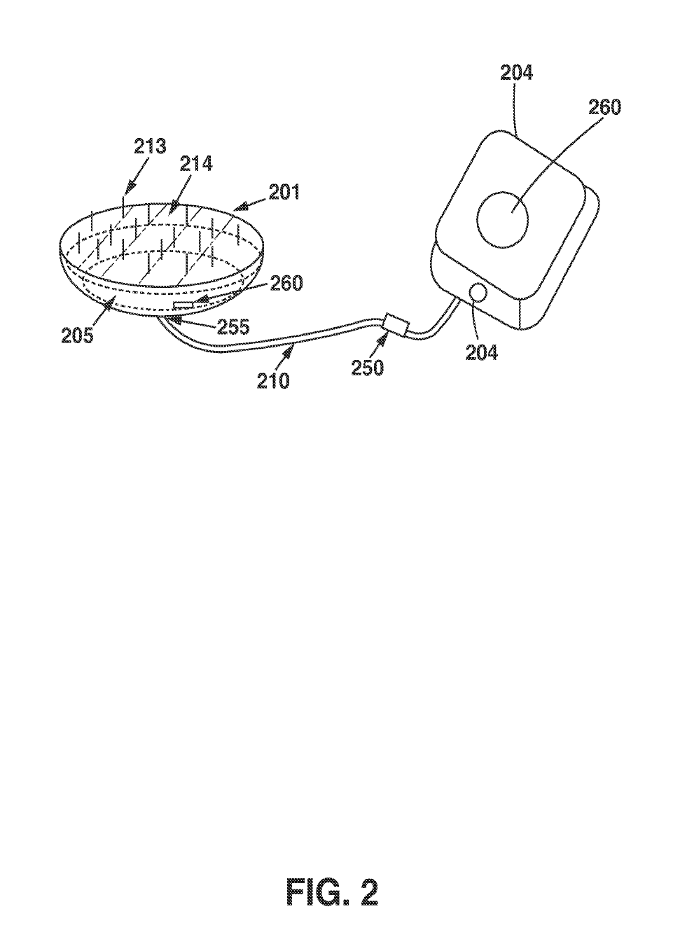

FIG. 2 shows an exemplary drug provisioning component 220 with an associated microneedle array. The drug provisioning component 220 contains a reservoir for storing the composition with the natriuretic peptide and a pumping mechanism. For chronic delivery, the reservoir (not shown) can be configured to hold up to several milliliters of the composition. Further, the drug provisioning component 220 in combination with the intradermal needle array can be carried on the person of the patient for an extended period of time wherein the drug provisioning component 220 can be adapted to be attached to an article of clothing remote from the site of drug administration.

The drug delivery component 220 for use in embodiments of the invention can be designed to be compact (e.g., less than 15 cm×15 cm) as well as water resistant for ease of transportation by the patient. The drug delivery component 220 is associated with a microneedle array where the composition under pressure is transported between the drug delivery component 220 having the pumping mechanism and the microneedle array via flexible tubing or catheter 210. The pump can be operated by the patient, wherein the patient presses a button 260, which causes the release of a predetermined volume of the drug. In other embodiments, delivery is performed automatically under computer control where the patient does not control delivery. In such configurations, a drug release button 260 is not present, but control and processing components (not shown) disposed within the drug delivery component 220 control a pumping mechanism to deliver a quantity of drug at a specific rate and duration.

The electronic circuitry employed in drug delivery component 220 can take any form known to those of ordinary skill. It will be understood that conventional components and circuitry such as digital clocks, power supply for powering the circuits and providing telemetry circuits for telemetry transmissions between the device and an external programmer (not shown) are contemplated by the invention. The drug delivery component 220 can be controlled by software, firmware and hardware means that cooperatively monitor the dosing regimen and determine when to deliver, increase, decrease or stop delivery of a drug. The device can also monitor and adjust the dose rate as required.

Examples of communication between the drug delivery component 220 and a remote device or system via a remote data communication network are described in U.S. application Ser. No. 11/414,160, entitled “Remote Monitoring for Networked Fluid Infusion Systems,” which is herein incorporated by reference. For example, instructions can be transmitted to the drug delivery component 220 via a computer network, pager network, cellular telecommunication network, satellite communication network. Additionally, a memory can be configured in the drug delivery component 220 to store instructions associated with predetermined blood pressure, fluid status and kidney status parameters. For example, a programmer can be in telemetric communication with drug delivery component 220 by an RF communication link. The communication link can be any appropriate RF link such as Bluetooth, WiFi, MICS, or as described in U.S. Pat. No. 5,683,432 “Adaptive Performance-Optimizing Communication System for Communicating with an Implantable Medical Device” incorporated herein by reference in its entirety.

In certain embodiments, the invention includes a telemetry circuit that enables programming by means of an external programmer (not shown) via a 2-way telemetry link. Uplink telemetry allows device status and diagnostic/event data to be sent to the external programmer. Downlink telemetry allows the external programmer to allow the programming of function and the optimization of therapy for a specific patient. Known programmers and telemetry systems suitable for use in the practice of the present invention are contemplated by the invention. Programmers can communicate with the drug delivery component 220 via a bi-directional radio-frequency telemetry link, so that the programmer can transmit control commands and operational parameter values to be received by the drug delivery component 220, so that the device can communicate diagnostic and operational data to the programmer. Programmers suitable for the purposes of practicing the present invention include the Models 9790 and CareLink programmers, commercially available from Medtronic, Inc., Minneapolis, Minn.

Various telemetry systems for providing the necessary communications channels between an external programming unit and the drug delivery component 220 have been developed and are well known in the art. Telemetry systems suitable for the present invention include U.S. Pat. No. 5,127,404, entitled “Telemetry Format for Implanted Medical Device”; U.S. Pat. No. 4,374,382, entitled “Marker Channel Telemetry System for a Medical Device”; and U.S. Pat. No. 4,556,063 entitled “Telemetry System for a Medical Device.”

As a result, medication can be delivered to the user with precision without significant restriction on the user's mobility or lifestyle in an automated manner. The compact and portable nature of the pump and/or monitor affords a high degree of versatility in using the device. The ideal arrangement of the pump can vary widely depending upon the user's size, activities, physical handicaps and/or personal preferences.

As shown in FIG. 2, a catheter 210 is attached to a substrate 201 having a microneedle array 213 as described above on a surface 214 thereof. The composition is pumped into an intra-substrate space 205 within the body of the substrate 201. The microneedle array is in fluid communication with the intra-substrate space 205 and by consequence with the reservoir in housing 220. As such, the composition having the natriuretic peptide is delivered through the microneedle array and into the intradermal space of the patient. The surface 214 of the substrate 201 can be secured to the patient's skin by an adhesive substance present on the surface 214 or the substrate 201 can be held in place by an elastic member or any other suitable means. Optionally, the substrate 201 can contain a spring-loaded retractable mechanism to assist in piercing the skin of the patient. Alternatively, the array 213 can penetrate the skin due to manual pressure applied to the substrate 201.

Due to the small dimensions of individual microneedles within the microneedle array 213, plural needles can be present to allow for a satisfactory delivery of volume over time without the use of excessive pressure to drive the delivery of the composition. However, it is advantageous for the rate of delivery through each needle with the microarray 213 to be substantially equal. As described, the provisioning component 220 delivers the composition into an intra-substrate space 205 accessible to all of the individual microneedles in the array 213. Blockage of any one of the individual needles of the array 213 can produce unequal delivery between individual microneedles. Alternatively, conditions may develop where the pressure in different regions of the intra-substrate space 205 can develop that can affect the uniformity of composition delivery since the delivery port 255 for the catheter 210 is at a discrete location. The presence of particulate material in the intra-substrate space 205 or the presence of air, which can be dangerous to the patient, can affect the uniform delivery of the composition.

Pressure sensors can be configured to detect conditions that can result in the uneven delivery of the composition and/or air present in the intra-substrate space 205. In particular, a pressure sensor 250 can sense the pressure within delivery catheter 210. The pressure sensor 250 can be present in the housing 220 or in-line with catheter 210. Due to the constant viscosity of the composition containing the natriuretic peptide and the known volume pumping rate of the drug provisioning component, the pressure within the catheter 210 can be in a predictable range wherein the system is primed and no significant blockage of the individual needles of the array 214 are present. A pressure reading outside of the expected range can serve as a signal to stop administration and troubleshoot the cause of abnormal pressure.

Uneven delivery of the natriuretic peptide may not always manifest in a significant pressure change within the catheter 210 as measured by pressure sensor 250 due to the plurality of microneedles in the array 214. Additional pressure sensors 270 can be configured in the intra-substrate space to indicate a pressure within the intra-substrate space. A difference in pressure between the first pressure sensor 250 and the second pressure sensor 270 can be monitored during operation wherein an unexpected change in the monitored pressure difference can indicate an interruption in even flow between individual needles of the array 213.

FIG. 3 shows an additional embodiment of the drug provisioning component 220 with associated substrate 201 and microneedle array 213. In FIG. 3, the distal end of the catheter 210 is divided into a plurality of attachment members 310 for interaction with a plurality of ports for introduction of the composition into the intra-substrate space 205. However, the plurality of attachment members 310 in FIG. 3 serve to distribute the pressure of the composition from the catheter 210 in an even manner throughout the intra-substrate space 205.

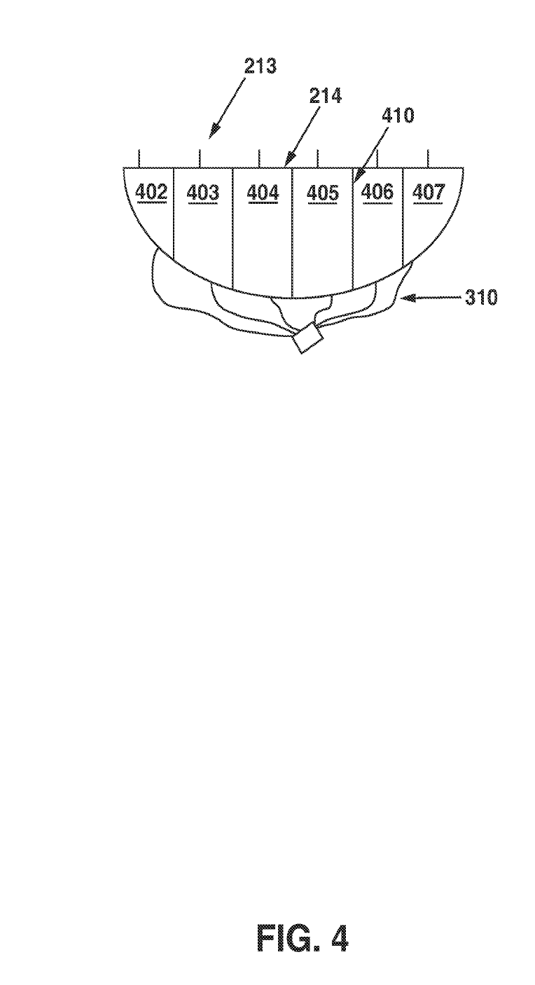

In certain embodiments, the intra-substrate space can be divided into one or more compartments 402, 403, 404, 405, 406 and 407 divided by a plurality of intra-substrate members 410 as shown in FIG. 4 In alternative embodiments, the plurality of intra-substrate space members 410 within the intra-substrate space may or may not define completely separate pressure areas wherein partial space members (not shown) define partially separate areas to assist in equilibrating pressures, but do not necessarily define areas of completely separate pressures. As shown in the present embodiment reflected in FIG. 4, the division of the intra-substrate space 205 into a plurality of separate compartments can minimize the development of pressure differences between different and separate pressure regions of the intra-substrate space since the volume over which pressure differences can develop is minimized. Further, the pressure in each of the plurality of attachment members 310 can be monitored to assess pressure equalization across the plurality of compartments 402-407. In certain embodiments, the substrate 201 with microneedle array 213 can be placed in a region near a superficial lymph node. For example, the microneedle array can be used to pierce the skin near the iliac, inguinual and femoral, popliteal, epitrochlear and brachial, supratrochlear and deltoideopectoral lymph nodes.

The pumps described herein and other similar or equivalent variants can be configured to deliver a dose of a natriuretic peptide to a patient using the systems, devices and methods of the present invention. Further, techniques related to infusion pump system operation, sensing and monitoring, signal processing, data transmission, signaling, network control, and other functional aspects of infusion pump and/or systems (and the individual operating components) are contemplated by the invention. Examples of infusion pumps and/or communication options may be of the type described in, but not limited to U.S. Pat. Nos. 4,562,751; 4,685,903; 5,080,653; 5,505,709; 5,097,122; 6,551,276, 6,554,798; 6,558,320; 6,558,351; 6,641,533; 6,423,035; 6,652,493; 6,656,148; 6,659,980; 6,752,787; 6,817,990; 6,872,200; 6,932,584; 6,936,029; 6,979,326; 6,997,920; and 7,025,743, which are herein incorporated by reference.

Examples of external infusion pumps include Medtronic MiniMed® Paradigm® pumps and one example of a suitable implantable pump is Medtronic SynchroMed® pump, all manufactured by Medtronic, Inc., Minneapolis, Minn. Another example of an implantable drug pump is shown in Medtronic, Inc. “SynchroMed® Infusion System” Product Brochure (1995). Additional examples of external infusion pumps include Animas Corporation Animas® and OneTouch® Ping® pumps, manufactured by Animas Corporation, Frazer, Pa. Implantable drug pumps can use a variety of pumping mechanism such as a piston pump, rotary vane pump, osmotic pump, Micro Electro Mechanical Systems (MEMS) pump, diaphragm pump, peristaltic pump, and solenoid piston pump to infuse a drug into a patient. Peristaltic pumps typically operate by a battery powered electric motor that drives peristaltic rollers over a flexible tube having one end coupled to a therapeutic substance reservoir and the other end coupled to an infusion outlet to pump the therapeutic substance from the therapeutic substance reservoir through the infusion outlet. Examples of solenoid pumps are shown in U.S. Pat. No. 4,883,467 “Reciprocating Pump For An Implantable Medication Dosage Device” to Franetzki et al. (Nov. 28, 1989) and U.S. Pat. No. 4,569,641 “Low Power Electromagnetic Pump” to Falk et al. (Feb. 11, 1986). An example of a pump is shown in U.S. Pat. No. 7,288,085 “Permanent magnet solenoid pump for an implantable therapeutic substance delivery device,” which is incorporated herein by reference. Further, the contents of U.S. Pat. App. Pub. No. 2008/0051716 directed to “Infusion pumps and methods and delivery devices and methods with same” is incorporated herein by reference. Additional examples of external pump type delivery devices are described in U.S. patent application Ser. No. 11/211,095, filed Aug. 23, 2005, titled “Infusion Device And Method With Disposable Portion” and Published PCT Application WO 2001/70307 (PCT/US01/09139), titled “Exchangeable Electronic Cards For Infusion Devices,” Published PCT Application WO 2004/030716 (PCT/US2003/028769), titled “Components And Methods For Patient Infusion Device,” Published PCT Application WO 2004/030717 (PCT/US2003/029019), titled “Dispenser Components And Methods For Infusion Device,” U.S. Patent Application Publication No. 2005/0065760, titled “Method For Advising Patients Concerning Doses Of Insulin,” and U.S. Pat. No. 6,589,229, titled “Wearable Self-Contained Drug Infusion Device,” each of which is incorporated herein by reference in its entirety. All such pumps can be adapted for external use to provide for intradermal delivery.

The drug delivery components and pumps used in the devices, systems and methods of the invention can have the desirable characteristics that are found, for example, in pumps produced and sold by Medtronic, such as Medtronic MiniMed® Paradigm® models. The Paradigm® pumps include a small, wearable control unit, which enables patients to program the delivery of the therapeutic agent via inputs and a display. The pump control unit includes microprocessors and software which facilitate delivery of the therapeutic agent fed from an included reservoir by a piston rod drive system. The pumps also include wireless telemetry for continuous system monitoring based on data obtained from optional sensors. Alternatively, continuous administration can be accomplished by, for example, another device known in the art, such as a pulsatile electronic syringe driver (e.g., Provider Model PA 3000, Pancretec Inc., San Diego Calif.), a portable syringe pump such as the Graseby model MS 16A (Graseby Medical Ltd., Watford, Hertfordshire, England), or a constant infusion pump such as the Disetronic Model Panomat C-S Osmotic pumps, such as that available from Alza, a division of Johnson & Johnson, may also be used. Since use of continuous intradermal injections allows the patient to be ambulatory, it is typically chosen over continuous intravenous injections.

Further examples of external pump type delivery devices are described in U.S. patent application Ser. No. 11/211,095, en titled “Infusion Device And Method With Disposable Portion” and Published PCT Application No. WO 2001/70307 (PCT/US01/09139), titled “Exchangeable Electronic Cards For Infusion Devices,” Published PCT Application No. WO 2004/030716 (PCT/US2003/028769), entitled “Components And Methods For Patient Infusion Device,” Published PCT Application No. WO 04/030717 (PCT/US2003/029019), entitled “Dispenser Components And Methods For Infusion Device,” U.S. Patent Application Publication No. 2005/0065760, entitled “Method For Advising Patients Concerning Doses Of Insulin,” and U.S. Pat. No. 6,589,229 entitled “Wearable Self-Contained Drug Infusion Device,” each of which is incorporated herein by reference in its entirety. The present invention contemplates the aforementioned pumps adapted for use in intradermally delivering natriuretic peptides via an array of microneedles.

In other embodiments, a pump can be disposed on the microneedle array to form a unitary structure that includes a control module connected to a fluid reservoir or an enclosed fluid reservoir that delivers the drug. The control module can include a pump mechanism for pumping fluid from the fluid reservoir to the patient. The control module can also include a pump application program for providing a desired therapy and patient specific settings accessed by the pump application program to deliver the particular desired therapy. The control module can optionally be connected or coupled or directly joined to a network element, node or feature that is communication with a database. In one embodiment, a communications port is provided to transfer information to and from the drug pump. Other embodiments include a wireless monitor and connections as described in U.S. Patent App. Pub. No. 2010/0010330, the contents of which are incorporated herein by their entirety. The pump can further be programmable to allow for different pump application programs for pumping different therapies to a patient as described herein.

The continuous pumps of the invention can be powered by gas or other driving means and can be designed to dispense drugs under pressure as a continual dosage at a preprogrammed, constant rate. The amount and rate of drug flow are regulated by the length of the catheter used, temperature, and are best implemented when unchanging, long-term drug delivery is required. The pumps of the invention preferably have few moving parts and require low power. Programmable pumps utilizing a battery-powered pump and a constant pressure reservoir to deliver drugs on a periodic basis can be programmed by the physician or by the patient. For a programmable infusion device, the drug may be delivered in small, discrete doses based on a programmed regimen, which can be altered according to an individual's clinical response. Programmable drug delivery pumps may be in communication with an external transmitter, which programs the prescribed dosing regimen, including the rate, time and amount of each dose, via low-frequency waves. Many drug delivery devices, implants and pumps of various configurations, in addition to those described herein, have been developed and are embraced by the present invention.

The rate of delivery of the therapeutic agent from the pump to the array of microneedles is typically controlled by a processor according to instructions received from a programmer. This allows for delivering similar or different amounts of the natriuretic peptide continuously, at specific times, or at set intervals between deliveries, thereby controlling the release rates to correspond with the desired targeted release rates. Typically, the pump is programmed to deliver a continuous or intermittent dose of a natriuretic peptide to prevent, or at least to minimize, fluctuations in natriuretic peptide serum or plasma level concentrations.

The pump can be configured or programmed to deliver the natriuretic peptide suitable for intradermal delivery in a constant, regulated manner for extended periods to avoid undesirable variations in blood-level drug concentrations. Generally, a pump can be distinguished from other diffusion-based systems in that the primary driving force for delivery by pump is pressure difference rather than concentration difference of the drug between the therapeutic formulation and the surroundings.

In some embodiments, the natriuretic peptides can be intradermally infused for pulsatile or intermittent periods of time where there are intervening time periods during which the natriuretic peptides are not administered or infused. For example, the natriuretic peptides can be intradermally infused for 4 hours on and 8 hours off, repeating for 3 days, at rates corresponding to an observed Cmax. This can generate an AUC that is approximately two times that of the single bolus injection but is comparable to an IV infusion AUC, considering that the IV infusion is to be given at 12 hour intervals.

In yet another embodiment, dosing can occur continuously at a rate that would match the AUC of a bolus intradermal injection. This can be accomplished where the total amount of natriuretic peptide infused can be reduced or the time frame can be limited similar to the second scenario. Alternatively, infusion may be performed for 2 hours on then 10 hours off, or following a similar schedule.

To maintain a plasma concentration of the natriuretic peptides within a specified range, a control module that controls or provides controlling instructions to the pump can be configured for use in the invention. The control module can adjust a dosing schedule and/or calculate a new dosing schedule for intradermal delivery via the array of microneedles. The control module can further contain a communications port to allow communication with the pump from another device located either locally or remotely relative to the pump. Further, memory configured either internally or externally to the pump housing can store various programs and data related to the operation of the pump. The memory is coupled to microprocessor, which, in turn, runs the desired operating programs which control operation of pump mechanism. Access to the microprocessor is provided through communications port or by other communication links such as infrared telemetry. Information programmed into memory instructs information to be transmitted or received via communications port or via infrared telemetry or other wireless means know to those of skill in the art. This feature allows information being received via communications port from an external device to control the pump. This feature also allows for the downloading of any or all information from memory to an external device.

It will be apparent to one skilled in the art that various combinations and/or modifications and variations can be made. Moreover, features illustrated or described as being part of one embodiment may be used on another embodiment to yield a still further embodiment.

### Example 1

### Simulation of Intradermal Delivery

Atrial natriuretic peptide (ANP) is reported to have limited bioavailability when administered as a subcutaneous (SQ) bolus, as reported by Crozier et al. and by Osterode et al. The mean terminal half-life of a 50 μg bolus of ANP is reported to be 3-fold greater when administered as an SQ bolus as compared to an intravenous (IV) bolus (p<0.05) by Osterode et al. The reduced bioavailability is likely caused by enzymatic degradation while the observed half-life after SQ bolus administration is likely due to the presence of a significant absorption half-life.

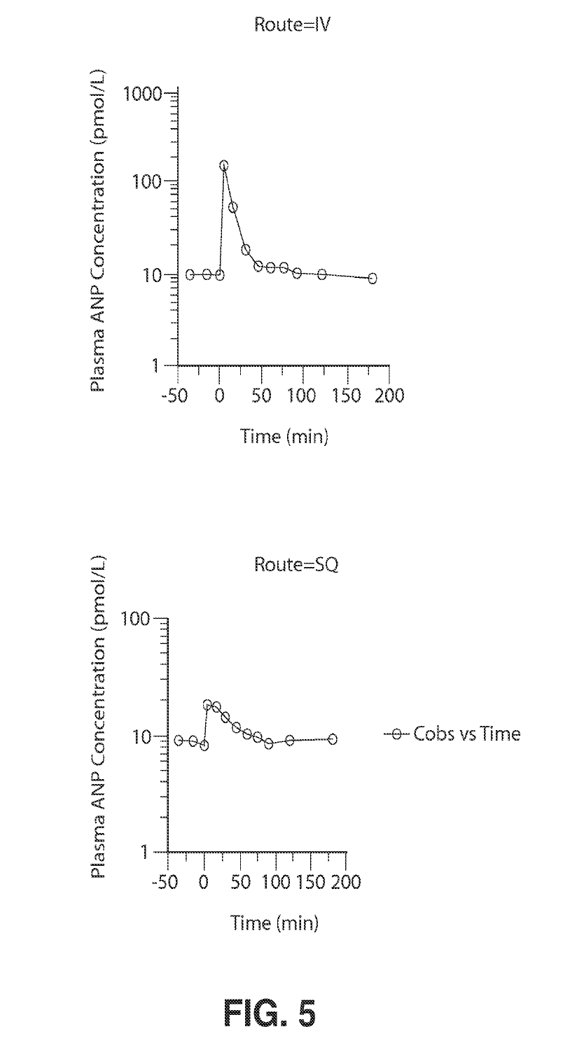

Osterode et al. published plasma concentration data for humans injected with ANP by either SQ or IV bolus. FIG. 5 presents the IV bolus data (FIG. 5A) and SQ bolus data (FIG. 5B) from the Osterode et al. with selected points digitized shown by open circles.

The digitized data points from FIG. 5A and FIG. 5B were baseline-corrected and analyzed using a commercial pharmacokinetic analysis software package (Phoenix v6.2, Pharsight, Cary, N.C.). Non-compartmental analysis (NCA) was performed first, followed by compartmental modeling. Bioavailability for administration into the SQ space (F) was estimated by dividing the area-under-the-curve (AUC) for the SQ bolus (FIG. 5B) by the AUC IV bolus (FIG. 5A), which was assumed to have 100% bioavailability. The slope of the terminal phase (λ_{Z}) for the IV and SQ data from FIG. 5 was estimated using linear regression. The estimate for λ_{Z} was converted to a half-life (t_{1/2}) using Equation 1.

t_{1/2}=ln(2)/λ_{z} (Eq. 1)

The half-life and λ_{Z} obtained from the regression analysis were used as an elimination half-life or elimination rate constant kₑ (1/min) to confirm that ANP behaves in a manner similar to a one-compartment model. For a one-compartment model of an IV bolus, the initial plasma concentration (C₀) after bolus injection is provided by Equation 2 while the change in plasma concentration (dC/dt) during the elimination phase is given by Equation 3.

$$\begin{matrix}{C}_{0} = \frac{{D}_{0}}{V} & ( Eq . 2 ) \\ \frac{d C}{d t} = - {k}_{e} C & ( Eq . 3 )\end{matrix}$$

Similarly, the one-compartmental model for SQ bolus injection can be modeled by Equations 4 and 5, where Aₐ is the amount of drug in the injection site (pmol), kₐ is the absorption rate constant (1/min), and F is the bioavailability as a unitless decimal. The initial estimates for primary model parameters kₑ and V/F were taken from the results of the NCA analysis of the IV data (kₑ=λ_{Z}) from the IV bolus data in FIG. 5A from Osterode et al. The initial estimate for the primary model parameter kₐ was taken from the results of the NCA analysis of the SQ data (kₐ=λ_{Z}) from the SQ bolus data in FIG. 5B. Clearance (CL) and t½ were calculated as secondary model parameters.

$$\begin{matrix}\frac{d {A}_{a}}{d t} = - {A}_{a} {k}_{a} & ( Eq . 4 ) \\ \frac{d C}{d t} = \frac{{Fk}_{a}}{V} {A}_{a} - {k}_{e} C & ( Eq . 5 )\end{matrix}$$

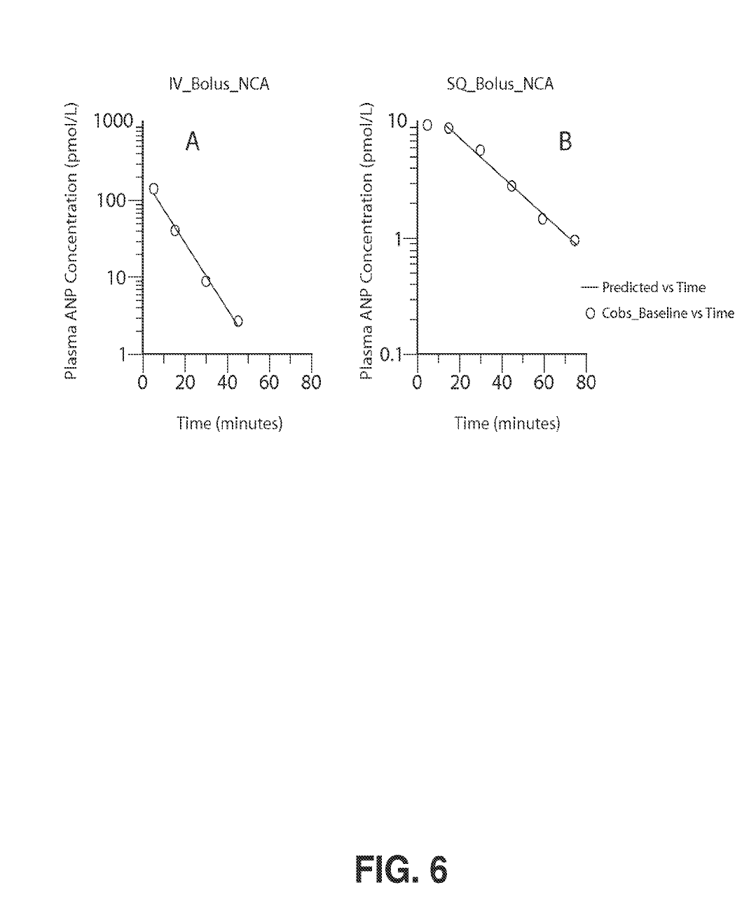

FIG. 6 shows the regression line generated from the digitized data for a 50 μg IV bolus (FIG. 6A) and SQ bolus (FIG. 6B). Table 1 reports the values for AUC, elimination t_{1/2}, volume of distribution (V) and clearance (CL) reported by Osterode et al. compared to those obtained from the NCA analysis of the digitized data as described above.

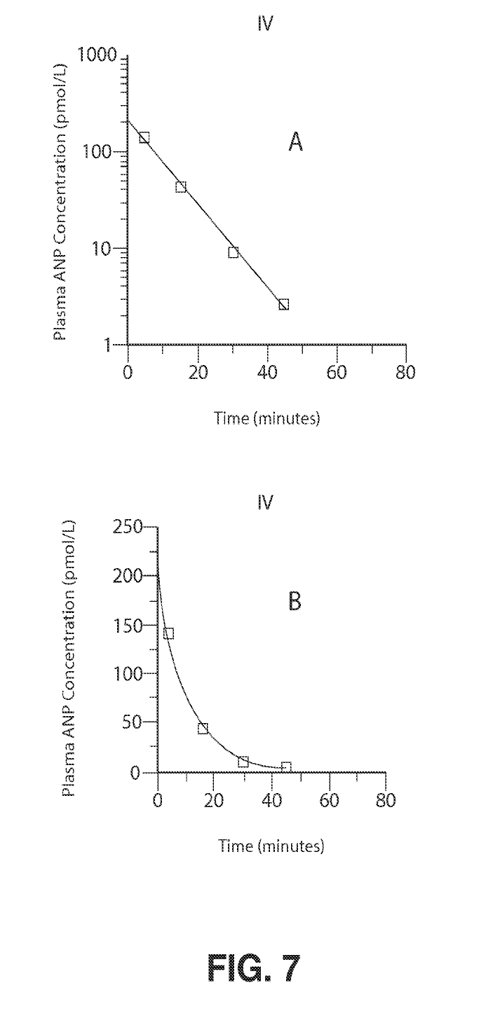

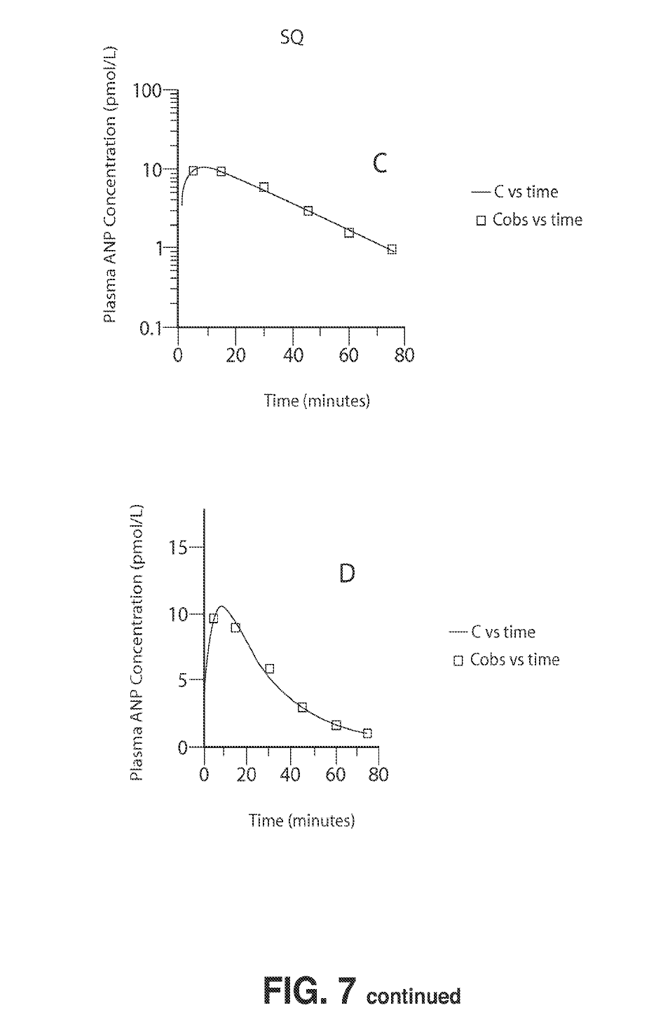

The results of the one-compartment modeling of the data from Osterode et al. are reported in Table 2 and FIG. 7, where the present NCA results are also included for comparison. FIGS. 7A (log scale) and 7B (non-log scale) show a model for a 50 μg IV bolus and FIGS. 7C (log scale) and 7D (non-log scale) show a model for a 50 μg SQ bolus. Both the NCA and compartmental analysis estimate the bioavailability to be approximately 20%. It is notable that the estimates for kₐ from the SQ bolus data are nearly identical for the NCA and compartmental analysis (0.039 min⁻¹), while the compartmental analysis estimate for kₑ is 2.6-times greater than the NCA estimate (0.26 vs. 0.099 min⁻¹). Hence, the NCA analysis estimates that elimination is 2.6-times faster than absorption (kₑ/kₐ=0.0991/0.0388=2.6), while the compartmental model estimates that elimination is 6.6-times greater than absorption (kₑ/kₐ=0.26/0.0394=6.6).

TABLE 1
Comparison of the PK parameters reported
by Osterode et al. and the results of the present NCA

| | IV | | | | SQ | | | | |
|---|---|---|---|---|---|---|---|---|---|
| | AUC | t_{1/2} | V | CL | AUC | t_{1/2} | V/F | CL/F | |
| Analysis | (min-pmol/L) | (min) | (L) | (L/min) | (min-pmol/L) | (min) | (L) | (L/min) | F |
| Osterode et al. | 1793 | 5.6 | NR | NR | 345 | 16.6 | NR | NR | 19.2% |
| (2) | | | | | | | | | |
| Present NCA | 1731 | 7.0 | 93.2 | 9.2 | 344 | 17.9 | NR | 44.1 | 19.9% |

TABLE 2
Comparison of the PK parameters calculated
by the present NCA and one-compartment analyses

| | IV | | | | SQ | | | | | |
|---|---|---|---|---|---|---|---|---|---|---|
| | V | kₑ | CL | t_{1/2} | kₐ | V/F | kₑ | CL/F | t_{1/2} | |
| Analysis | (L) | (l/min) | (L/min) | (min) | (l/min) | (L) | (l/min) | (L/min) | (min) | F |
| Present NCA | 93.2 | 0.0991 | 9.2 | 7.0 | 0.0388 | NR | NA | 44.1 | 17.9 | 19.9% |
| 1-Compartment Model | 79.5 | 0.0990 | 7.9 | 7.0 | 0.0394 | 167 | 0.26 | 43.3 | 17.6 | 18.2% |

The potential ability of intradermal delivery to enhance the pharmacokinetics of ANP was explored using the 1-compartment model (Equations 4-5) to simulate two scenarios for bolus administration by intradermal delivery. In the first scenario, it was assumed that intradermal delivery will have a significantly increased absorption rate (kₐ>>kₑ). Such an increase in absorption rate will effectively eliminate any loss of ANP to enzymatic degradation or receptor uptake prior to entry into the blood stream (F=100%). As shown in Table 2, kₐ was calculated for a 1-compartmental model to be 0.0394 min⁻¹. To show the effect of a significantly increased absorption rate, the pharmacokinetic profile of ANP was modeled with a kₐ of 100 min⁻¹ while maintaining the value of the elimination rate constant (kₑ) at 0.0990 min⁻¹. For the second scenario, it was assumed that intradermal delivery will not accelerate the adsorption rate, but that the intradermal space will have negligible enzymatic activity and/or receptor density such that all of the injected ANP will be bioavailable (F=100%). The values of the model parameters used for these two simulations are provided in Table 3.

TABLE 3
Model parameter values used for the simulation
of the bolus administration of ANP to the ID space.

| | Scenario 1: ID | Scenario 2: ID Bolus, |
|---|---|---|
| Model Parameter | Bolus, Fast Absorption | Unrestricted Bioavailability |
| Intradermal Bolus Dose | 50 | 50 |
| (mcg) | | |
| kₐ (1/min) | 100.0 (kₐ>>kₑ) | 0.0394 |
| V (L) | 79.5 | 79.5 |
| kₑ (1/min) | 0.0990 | 0.0990 |
| F | 100% | 100% |

The potential ability of intradermal delivery to enhance the pharmacokinetics of ANP was further explored using the 1-compartment model (Equations 4-5) to simulate three scenarios for the continuous infusion of ANP at a rate of 1.33 μg/min. In the first scenario, the continuous infusion of ANP into the SQ space was considered as a base case. In the second scenario, the continuous infusion of ANP into the intradermal space was considered, assuming fast adsorption into the blood stream. In the third scenario, the continuous infusion of ANP into the intradermal space was considered, assuming unrestricted bioavailability. The model parameters used to create a simulation for SQ infusion (Scenario 1) and for ID infusion with the fast absorption rate constant (Scenario 2) and for ID infusion with the normal absorption rate constant but with 100% bioavailability (Scenario 3) are shown in Table 4.

TABLE 4
Model parameter values used for the simulation of
the continuous infusion of ANP to the SQ and ID space

| | Scenario 1: | Scenario 2: ID | Scenario 3: ID Infusion, |
|---|---|---|---|
| Model Parameter | SQ Infusion | Infusion, Fast Absorption | Unrestricted Bioavailability |
| ANP Infusion Rate (μg/min) | 1.33 | 1.33 | 1.33 |
| kₐ (1/min) | 0.0394 | 100.0 (kₐ>>kₑ) | 0.0394 |
| V (L) | 30.4 | 79.5 | 79.5 |
| kₑ (1/min) | 0.26 | 0.0990 | 0.0990 |
| F | 18.2% | 100% | 100% |

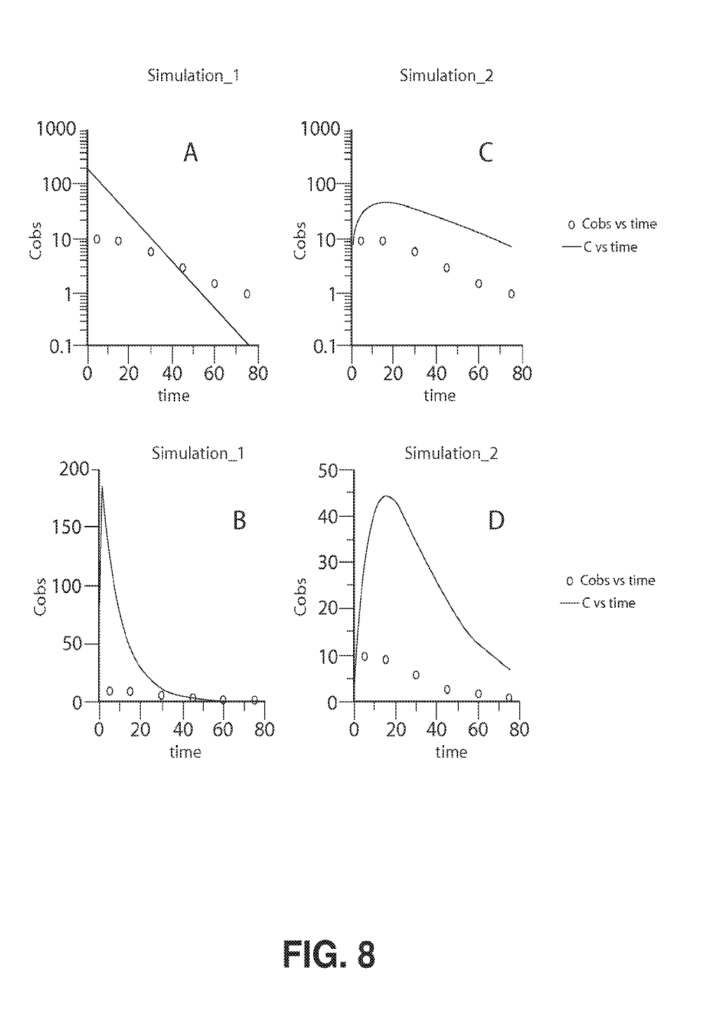

The results of the two pharmacokinetic simulations of the intradermal bolus delivery of ANP are provided in FIG. 8 in accordance with Scenarios 1 and 2 described above. FIGS. 8A and 8B show the Scenario 1 ID bolus simulation with a log y-axis and non-log y-axis, respectively, with the digitized observed SQ data from Osterode et al. shown in open circles. The simulation of the fast-absorption scenario (Scenario 1, FIGS. 8A and 8B) predicts that intradermal delivery will increase Cₘₐₓ by an approximate factor of 17 compared to SQ delivery. The fast-absorption simulation looks similar to the model and data for the IV bolus shown in 7A. The simulation results also illustrate that intradermal delivery would increase the ANP AUC by a factor of 5 under the fast-absorption scenario. FIGS. 8C and 8D shows the Scenario 2 ID bolus simulation with a log y-axis and non-log y-axis, respectively, with the digitized observed SQ data from Osterode et al. shown in open circles. The simulation of the unrestricted bioavailability scenario (Scenario 2) predicts that intradermal delivery will increase Cₘₐₓ by an approximate factor of 4.5 compared to SQ delivery. The simulation results also illustrate that intradermal delivery would increase ANP AUC by a factor of 5 under the unrestricted bioavailability scenario.

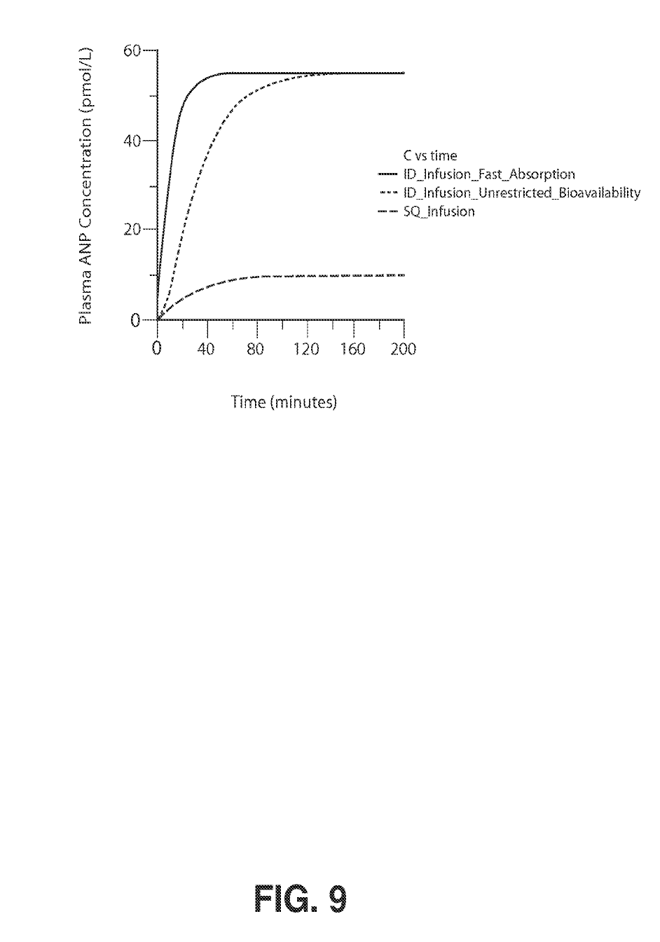

The results of the 3 pharmacokinetic simulations of the continuous infusion of ANP are provided in FIG. 9. Here, the steady-state concentrations of ANP are predicted to be approximately 5.5-times greater for ID vs. SQ administration. Furthermore, the ID scenario that assumes fast absorption approaches steady-state faster than the ID scenario that assumes unrestricted bioavailability.

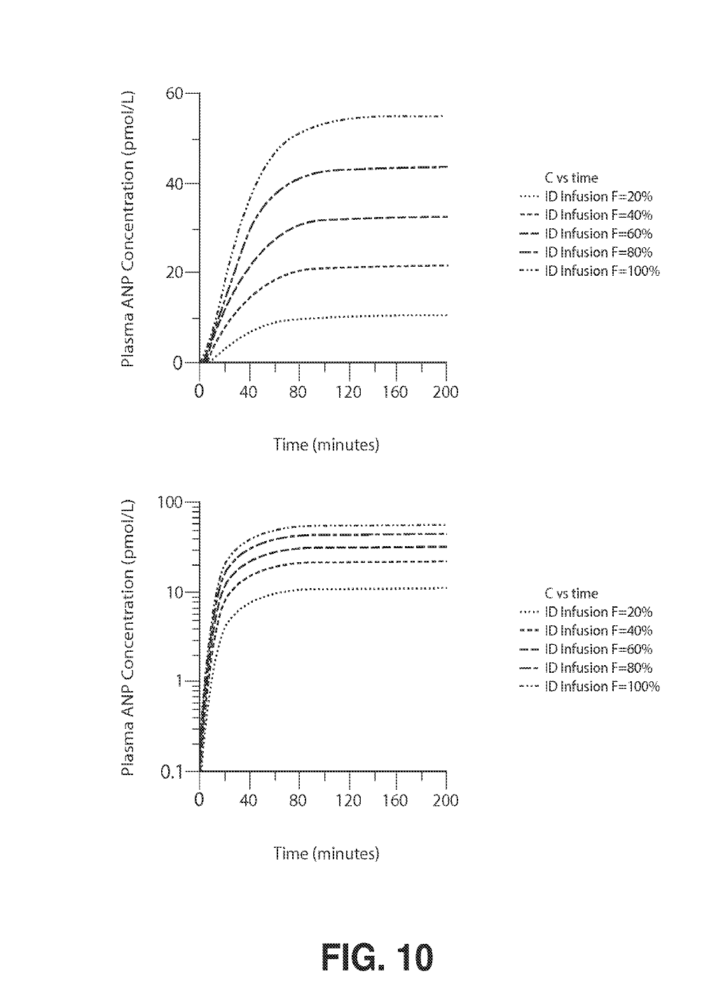

As discussed, FIG. 9 presents a simulation of ANP at a rate of 1.33 μg/min for a subject exhibiting unrestricted 100% bioavailability with an absorption rate constant (kₐ) of 0.0394 min⁻¹. FIG. 10 presents the effect of bioavailability less than 100% with other pharmacokinetic parameters unmodified. As shown in FIG. 10, the AUC and the steady state concentration reached during infusion is directly proportional to the bioavailability. It should be noted that in system where bioavailability (F) is 100%, a change in the absorption rate constant (kₐ) that may be observed between individual patients would not affect the steady state concentration reached; however, a decrease in kₐ (increase in absorption half-life) would increase the time needed to reach a steady state condition for plasma concentration and thereby affect the AUC from 0 to 200 minutes. An increase in the kₐ would decrease the time to reach steady state. However, where bioavailability is less than 100% and the absorption half-life includes a kₐ₂ rate constant, the F equals kₐ₁/(kₐ₁+kₐ₂), where kₐ=kₐ₁+kₐ₂, and the area under the curve (from 0 minutes to infinity as well as 0 minutes to 200 minutes) and the steady state concentration will be affected by a change in absorption half-life (change in kₐ₂ results in change in kₐ). An increase in elimination rate constant (kₑ) (or an increase in clearance, since clearance=kₑ×VOD) would have a tendency to decrease the steady state plasma concentration, which may necessitate an increased dosage to maintain a higher steady state concentration.

The results of the NCA (Table 1) and one-compartment (Table 2) modeling of the human PK data published by Osterode et al. confirm their reported estimates for terminal half-life, AUC, and bioavailability of ANP administered as a 50 μg bolus to the intravenous and subcutaneous spaces. The simulation of various pharmacokinetic scenarios illustrates the potential of intradermal delivery to improve the pharmacokinetics of ANP relative to subcutaneous delivery.

### Example 2

### Confirmation of Peptide Bioavailability

Due to the similarity of porcine skin to human skin, pigs can be tested to provide confirmation that bioavailability is increased by an intradermal delivery route compared to a SQ delivery route. Two vascular access ports can be implanted to enable IV peptide (such as BNP) infusions and blood sample collection at various time points prior to and following drug administration. Using measured blood serum or plasma drug levels, IV, SQ and ID bioavailability can be directly compared. Blood can be drawn at multiple time points (for example, 10 min prior to drug infusion and at 1, 5, 10, 20, 30, 40, 50, 60, 90, 120, 180, and 240 min post-infusion) from each pig for each route of drug delivery. As shown in Table 5, 3 separate animals can be used in a rotation such that each mode of delivery is evaluated on each of the 3 animals. A day of recovery will be included between each drug administration to allow physiological equilibration (e.g. feeding, drug washout) between tests.

TABLE 5
Delivery Rotation

| Animal | Day 1 | Day 3 | Day 5 |
|---|---|---|---|
| 1 | IV | TD (Left) | SubQ (Right) |
| 2 | SubQ (Left) | IV | TD (Right) |
| 3 | TD (Right) | SubQ (Left) | IV |

Peptide drug plasma levels can be measured by enzyme immunoassay. The data can be fit with PK models to determine drug PK and bioavailability (calculated as the area under the curve) properties following the intradermal and subcutaneous routes of administration and compared to the intravenous infusion where the peptide is assumed to be the 100% bioavailable. To monitor and document the intradermal and subcutaneous infusion sites, digital pictures of the skin surface will be taken at the time of blood draws. Furthermore, tissue will be harvested post-mortem to examine the local tissue reaction to the drug following the different delivery methods.

### Example 3

### Confirmation of Peptide Stability

To provide a confirmation of the relative stability of a natriuretic peptide (such as BNP or other natriuretic peptides) in lymph fluid compared to serum plasma, an ex vivo experiment can be performed incubating BNP (or another natriuretic peptide) in blood, lymph fluid and a phosphate-buffered saline (PBS) control and comparing peptide half-lives in each of the fluids. Two concentrations of BNP (or another natriuretic peptide) (e.g., 1000 pg/ml and 100 pg/ml) can be incubated in blood plasma, lymph fluid or PBS at 37° C. for varying times. For example, time points collected may include 0, 1, 5, 10, 20, 30, 40, 50, 60, 90 and 120 minutes. Peptidase activity will be stopped by the addition of a protease inhibitor cocktail at each collection point. The concentration of BNP (or another natriuretic peptide) in each time point sample can be evaluated using an enzyme-linked immune assay (ELISA).

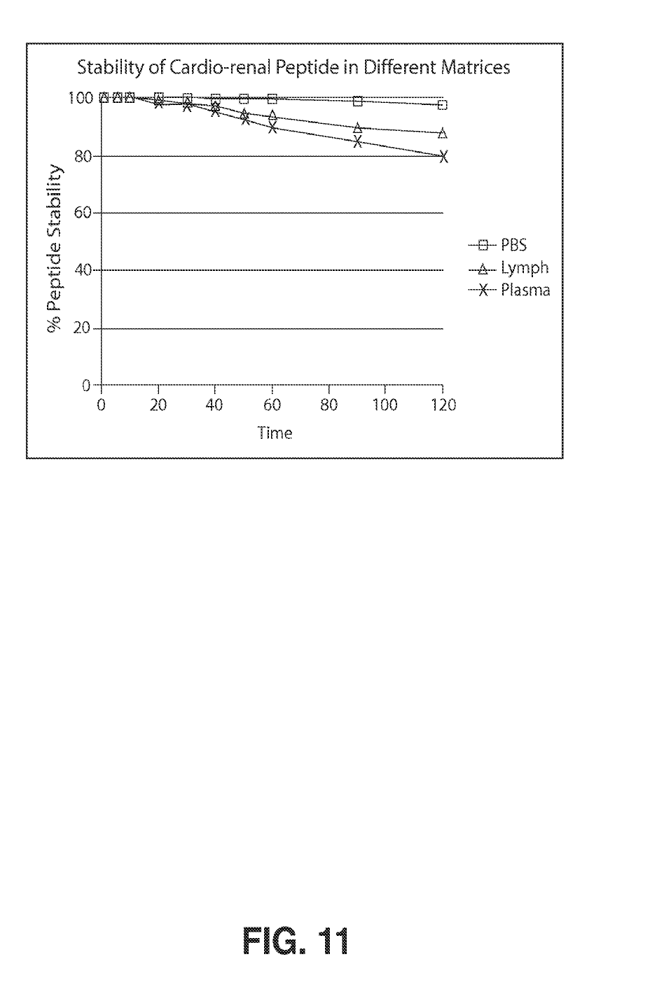

FIG. 11 shows hypothetical data for the stability study described in this Example. PBS, as the control, is expected to show minimal degradation of the peptide over time.

### Example 4

### Pharmacokinetic Parameters from Simulations of Example 1

The value of observed PK parameters can vary from person-to-person. Subjects can vary in the absorption parameters in particular and can vary from those reported in the simulations of Example 1. In certain embodiments, a subject can exhibit a half-life for absorption of a natriuretic peptide from 0 to 60 minutes depending upon the physiological state of the subject. The absorption half-life can be described by the range of n to (n+i) minutes, where n={xε [symbol] |0<x≦(60−n)}, and i={yε [symbol] |0≦y≦(60−n)}. In certain other embodiments, a subject can exhibit a half-life for intradermal absorption of the peptide from 0 to about 30 minutes, from 0 to about 5 minutes, from about 15 to 25 minutes, and from about 15 to about 30 minutes, in addition to about 20 minutes.

Subjects can further vary in elimination parameters for removal of the peptide, depending upon the physiological state of the patient and other additional parameters. In certain embodiments, a subject can exhibit a half-life for elimination of the natriuretic peptide from about 1 minute to about 45 minutes, from about 2 minutes to about 40 minutes, from about 3 minutes to about 30 minutes, from about 1 minute to about 20 minutes or from about 3 minutes to about 35 minutes. In a further embodiment, a subject can exhibit a half-life for elimination of the peptide from about 1 minutes to about 45, as described by the range of n to (n+i) minutes, where n={xε [symbol] |1≦x≦45} and i={yε [symbol] |0≦y≦(45−n)}.

The half-life for elimination of the peptide can have a noticeable effect on the pharmacokinetics exhibited for the peptides described herein. As discussed herein, a subject can exhibit a half-life for elimination falling into one of several ranges. Half-life for elimination is believed to be impacted by the physiological state of the subject. This includes not only the weight, age, water-retention of the subject, but also the presence of specific disease states, including impairment of kidney function. A subject can have kidney impairment such that the glomerular filtration rate is less than about 60 mL/min/1.73 m², as represented by the range from n to (n+i) mL/min/1.73 m², where n={xε [symbol] |0<x≦60} and i={yε [symbol] |0≦y≦(60−n)}. In certain other embodiments, a subject has a glomerular filtration rate less than about 15 mL/min/1.73 m² or in the range from 0 to about 60 mL/min/1.73 m². It is believed that subjects exhibiting impairment of kidney function and/or kidney disease may sometimes unexpectedly display a shorter half-life for elimination of natriuretic peptides compared to individuals not having kidney disease. That is, it is expected that subjects having kidney disease would have a longer half-life for elimination of the peptide compared with the average healthy individual not displaying impairment of kidney function.

In certain embodiments, the maximum plasma concentration or steady state concentration achieved by infusion of the peptide by intradermal infusion is from about 5 to about 200 pmol/L, as described herein. In certain other embodiments, the steady state plasma concentration achieved by intradermal infusion can be from about 10 to about 150 pmol/L, about 5 to about 100 pmol/L, from about 10 to about 75 pmol/L, from about 5 to about 55 pmol/L, from about 10 to about 60 pmol/L, from about 5 to about 40 pmol/L or from about 5 to about 50 pmol/L. In additional embodiments, the steady state plasma concentration achieved by intradermal infusion can be from more than 0 to about 55 pmol/L, from about 0.5 to about 55 pmol/L, from about 2 to about 55 pmol/L or from about 5 to about 55 pmol/L.

The maximum plasma concentration or steady state plasma concentration achieved by infusion is influenced by the rate of infusion or dosing administered to the subject. In certain embodiments, the peptide is administered by intradermal infusion at a rate from about 0.1 to about 10 μg/min of a natriuretic peptide in certain embodiments. In other embodiments, a subject can require an intradermal infusion dose from about 0.5 to about 10 μg/min, from about 1 to about 10 μg/min or from about 1 to about 5 μg/min.

1. An intradermal delivery device, comprising:
a drug provisioning component having a pumping apparatus to administer a therapeutically effective amount of a therapeutic composition from a reservoir;
a microneedle array having a substrate with plural microneedles projecting from a surface of the substrate, the microneedles in fluid communication with the reservoir and an intra-substrate space;
a catheter for transporting the composition from the reservoir to the intra-substrate space;
a first pressure sensor for sensing a pressure within the catheter; and
a controller for controlling a pumping rate of the pump and for monitoring a pressure within the catheter to determine if flow through the microneedle array is within an expected range.

2. The device of claim 1, further comprising a second pressure sensor for sensing a pressure within the intra-substrate space, wherein the controller monitors a pressure difference between the first pressure sensor and the second pressure sensor and determines if the flow rate between individual microneedles of the microneedle array is substantially equal.

3. The device of claim 1, wherein the therapeutic composition comprises one or more natriuretic peptides selected from any one of long-acting natriuretic peptide (LANP), kaliuretic peptide (KP), urodilatin (URO), brain natriuretic peptide (BNP), atrial natriuretic peptide (ANP), and vessel dilator (VD).

4. The device of claim 1, wherein the therapeutic composition comprises one or more chimeric natriuretic peptides selected from any one of CD-NP (SEQ ID No. 9), CU-NP (SEQ ID No. 10), DNP (SEQ ID No. 8), and CNP (SEQ ID No. 7).

5. The device of claim 3, wherein the composition has a concentration of the natriuretic peptide from 0.5 to 10 mg/mL.

6. The device of claim 1, wherein the drug provisioning component administers the composition at a rate from 1 to 100 μL/min.

7. The device of claim 1, wherein a distal end of the catheter for attachment to the intra-substrate space is divided into plural attachment members, the plural attachments attached to separate ports on the microneedle array

8. The device of claim 1, wherein the intra-substrate space is divided into one or more compartments.

9. The device of claim 1, wherein one or more intra-substrate space members are disposed within the intra-substrate space to reduce the volume of the intra-substrate space.

10. The device of claim 3, wherein the drug provisioning component delivers a therapeutically effective amount of the natriuretic peptide at a rate (ng/kg of body weight) from any one of 0.5 to 10 μg/min, from 1 to 10 μg/min or from 1 to 5 μg/min.

11. The device of claim 3, wherein the drug provisioning component delivers a therapeutically effective amount of the natriuretic peptide to maintain a plasma level of the natriuretic peptide at a steady state concentration from 0.5 to 200 pmol/L.

12. The device of claim 3, wherein the drug provisioning component delivers a therapeutically effective amount of the natriuretic peptide to maintain a plasma level of the natriuretic peptide at a steady state concentration or maximum plasma concentration in the range represented by n to (n+i) pmol/L, where n={xεZ|0<x≦200} and i={ yεZ|0≦y≦(200−n)}.

13. The device of claim 3, wherein the drug provisioning component delivers a therapeutically effective amount of the natriuretic peptide to maintain a plasma level of the natriuretic peptide at a steady state concentration or maximum plasma concentration from any one of 10 to 150 pmol/L, 5 to 100 pmol/L, from 10 to 75 pmol/L, from 5 to 55 pmol/L, from 10 to 60 pmol/L, from 5 to 40 pmol/L or from 5 to 50 pmol/L, from more than 0 to 55 pmol/L, from 0.5 to 55 pmol/L, from 2 to 55 pmol/L or from 5 to 55 pmol/L.

14. The device of claim 1, wherein the microneedles have a length selected from any of 300 to 1500 μm, from 500 to 900 μm, from 200 to 1200 μm, from 300 to 1000 μm, from 400 to 900 μm, from 600 to 800 μm and from 700 to 900 μm.

15. The device of claim 3, wherein the drug provisioning component delivers a composition comprising the natriuretic peptide at a rate selected from any of 1 to 200 μL/min, 5 to 150 μL/min, 3 to 100 μL/min, from 1 to 50 μL/min, from 1 to 75 μL/min, from 1 to 20 μL/min, from 1 to 15 μL/min and from 1 to 10 μL/min.

16. The device of claim 1, wherein the drug provisioning component delivers the therapeutic composition at a fixed, pulsed, continuous or variable rate.

17. The device of claim 1, wherein the drug provisioning component delivers an intermittent bolus of the therapeutic composition.

18. A method, comprising the steps of:
administering a therapeutic composition by intradermal administration to a patient suffering from kidney disease alone, heart failure, concomitant kidney disease and heart failure, or cardiorenal syndrome using a drug provisioning component, and
maintaining a plasma concentration of the composition within a specified range, wherein the bioavailability of the natriuretic peptide is increased or the half-life of absorption of the composition is decreased as compared to the composition delivered by subcutaneous administration.

19. The method of claim 18, wherein the drug provisioning component has a pumping apparatus to administer an amount of the composition from a reservoir, the drug provisioning component in fluid communication with a microneedle array having a substrate with plural microneedles projecting from a surface of the substrate, the microneedles in fluid communication with the reservoir and an intra-substrate space;
transporting the composition from the reservoir to the intra-substrate space with a catheter;
sensing a first pressure within the catheter using a first pressure sensor; and
monitoring a pressure within the catheter to determine if flow through the microneedle array is within an expected range.

20. The method of claim 19, further comprising sensing a second pressure within the intra-substrate space using a second pressure sensor and monitoring a difference between the first pressure and the second pressure and determining if the flow rate between individual microneedles of the microneedle array is substantially equal.

21. The method of claim 18, wherein the composition is a natriuretic peptide selected from any one of long-acting natriuretic peptide (LANP), kaliuretic peptide (KP), urodilatin (URO), atrial natriuretic peptide (ANP), brain natriuretic peptide (BNP) and vessel dilator (VD).

22. The method of claim 18, wherein the composition comprises one or more chimeric natriuretic peptides selected from any one of CD-NP (SEQ ID No. 9), CU-NP (SEQ ID No. 10), DNP (SEQ ID No. 8), and CNP (SEQ ID No. 7).

23. The method of claim 18, wherein the drug provisioning component is capable of delivering the natriuretic peptide at a fixed, pulsed, continuous or variable rate.

24. The method of claim 18, further comprising the step of collecting data and transmitting the data via radio frequency to an external controller.

25. The method of claim 18, further comprising the step of collecting and transmitting data and returning digital instructions to a control unit via the Internet.

26. The method of claim 18, wherein the drug provisioning component and a control unit are connected or controlled wirelessly.

27. The method of claim 19, wherein the microneedles have a length selected from any of from 300 to 1500 μm, from 500 to 900 μm, from 200 to 1200 μm, from 300 to 1000 μm, from 400 to 900 μm, from 600 to 800 μm and from 700 to 900 μm.

28. The method of claim 18, wherein the drug provisioning component delivers a composition comprising the composition at a rate selected from any of 1 to 200 μL/min, 5 to 150 μL/min, 3 to 100 μL/min, from 1 to 50 μL/min, from 1 to 75 μL/min, from 1 to 20 μL/min, from 1 to 15 μL/min and from 1 to 10 μL/min.

29. The method of claim 18, wherein the composition comprises brain natriuretic peptide (BNP).
